(19) United States
(12) Patent Application Publication
(10) Pub. No.: US 2024/0287028 A1
(43) Pub. Date: Aug. 29, 2024

(54) HERBICIDAL PHENYLURACILS

(71) Applicant: BASF SE, Ludwigshafen (DE)

(72) Inventors: Tobias Seiser, Ludwigshafen (DE); Matthias Witschel, Ludwigshafen (DE); Desislava Slavcheva Petkova, Ludwigshafen (DE); Michael Betz, Ludwigshafen (DE); Trevor William Newton, Limburgerhof (DE); Liliana Parra Rapado, Limburgerhof (DE); Ricardo Hugo Pavon Romero, Dos Hermanas (ES)

(21) Appl. No.: 18/578,350

(22) PCT Filed: Jul. 6, 2022
(86) PCT No.: PCT/EP2022/068674

§ 371 Date: Jan. 11, 2024

(30) Foreign Application Priority Data
Jul. 16, 2021 (EP) 21185972.3

(51) Int. Cl. C07D 401/12; A01N 43/54; A01P 13/00; C07D 239/54

(52) U.S. Cl. CPC C07D 401/12; A01N 43/54; A01P 13/00; C07D 239/54

(57) ABSTRACT

The present invention relates to phenyluracils of formula (I) or their agriculturally acceptable salts or derivatives, wherein the variables are defined according to the description, compositions comprising them and their use as herbicides. i.e. for controlling harmful plants, and also a method for controlling unwanted vegetation which comprises allowing a herbicidal effective amount of at least one phenyluracil of formula (I) to act on plants, their seed and/or their habitat.

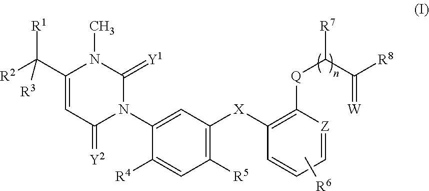

The present invention relates to phenyluracils of formula (I) defined below and to their use as herbicides.

EP 1 122 244, EP 1 106 607 and WO 19/101551 disclose similar compounds for which herbicidal action is stated, however only compounds, wherein the uracil is substituted by a trifluoromethyl group, are disclosed explicitly.

The herbicidal properties of these known compounds regarding the undesired vegetation are not always entirely satisfactory.

It is therefore an object of the present invention to provide phenyluracils of formula (I) having improved herbicidal action. To be provided are in particular phenyluracils of formula (I) which have high herbicidal activity, in particular even at low application rates, and which are sufficiently compatible with crop plants for commercial utilization.

These and further objects are achieved by phenyluracils of formula (I), defined below, and by their agriculturally suitable salts.

Accordingly, the present invention provides phenyluracils of formula (I)

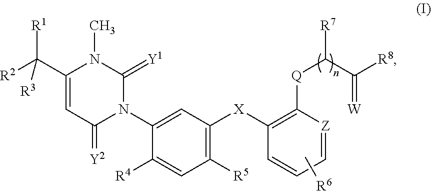

wherein the substituents have the following meanings:
R¹ CH₃;
R² hydrogen, halogen, C₁-C₄-alkyl, C₁-C₄-haloalkyl, C₁-C₄-alkoxy or C₁-C₄-haloalkoxy;
R³ hydrogen, halogen, C₁-C₄-alkyl, C₁-C₄-haloalkyl, C₁-C₄-alkoxy or C₁-C₄-haloalkoxy;
R⁴ H or halogen;
R⁵ halogen, CN, NH₂, NO₂;
R⁶ H, halogen, C₁-C₃-alkyl, C₁-C₃-alkoxy;
R⁷ H, C₁-C₃-alkyl, C₁-C₃-alkoxy;
R⁸ OR⁹, SR⁹, NR¹⁰R¹¹, NR⁹OR⁹, NR⁹S(O)₂R¹⁰ or NR⁹S(O)₂NR¹⁰R¹¹, wherein
R⁹ is hydrogen, C₁-C₆-alkyl, C₃-C₆-alkenyl, C₃-C₆-alkynyl, C₁-C₆-haloalkyl, C₃-C₆-haloalkenyl, C₃-C₆-haloalkynyl, C₁-C₆-cyanoalkyl, C₁-C₆-alkoxy-C₁-C₆-alkyl, C₁-C₆-alkoxy-C₁-C₆-alkoxy-C₁-C₆-alkyl, di(C₁-C₆-alkoxy)C₁-C₆-alkyl, C₁-C₆-haloalkoxy-C₁-C₆-alkyl, C₃-C₆-alkenyloxy-C₁-C₆-alkyl, C₃-C₆-haloalkenyloxy-C₁-C₆-alkyl, C₃-C₆-alkenyloxy-C₁-C₆-alkoxy-C₁-C₆-alkyl, C₁-C₆-alkylthio-C₁-C₆-alkyl, C₁-C₆-alkylsulfinyl-C₁-C₆-alkyl, C₁-C₆-alkylsulfonyl-C₁-C₆-alkyl, C₁-C₆-alkylcarbonyl-C₁-C₆-alkyl, C₁-C₆-alkoxycarbonyl-C₁-C₆-alkyl, C₁-C₆-haloalkoxycarbonyl-C₁-C₆-alkyl, C₃-C₆-alkenyloxycarbonyl-C₁-C₆-alkyl, C₃-C₆-alkynyloxycarbonyl-C₁-C₆-alkyl, amino, (C₁-C₆-alkyl)amino, di(C₁-C₆-alkyl)amino, (C₁-C₆-alkylcarbonyl)amino, amino-C₁-C₆-alkyl, (C₁-C₆-alkyl)amino-C₁-C₆-alkyl, di(C₁-C₆-alkyl)amino-C₁-C₆-alkyl, aminocarbonyl-C₁-C₆-alkyl, (C₁-C₆-alkyl)aminocarbonyl-C₁-C₆-alkyl, di(C₁-C₆-alkyl)aminocarbonyl-C₁-C₆-alkyl,
   —N═CR¹²R¹³, wherein R¹² and R¹³ independently of one another are H, C₁-C₄-alkyl or phenyl;
   C₃-C₆-cycloalkyl, C₃-C₆-cycloalkyl-C₁-C₆-alkyl, C₃-C₆-heterocyclyl, C₃-C₆-heterocyclyl-C₁-C₆-alkyl, phenyl, phenyl-C₁-C₄-alkyl or a 5- or 6 membered heteroaryl,
      wherein each cycloalkyl, heterocyclyl, phenyl or heteroaryl ring can be substituted by one to four substituents selected from R¹⁴ or a 3- to 7-membered carbocyclus,
         which carbocyclus optionally has in addition to carbon atoms one or two ring members selected from the group consisting of —N(R¹²)—, —N═N—, —C(═O)—, —O— and —S—, and
         which carbocyclus is optionally substituted with one to four substituents selected from R¹⁴;
         wherein R¹⁴ is halogen, NO₂, CN, C₁-C₄-alkyl, C₁-C₄-haloalkyl, C₁-C₄-alkoxy or C₁-C₄-alkoxycarbonyl;
R¹⁰, R¹¹ independently of one another are R⁹, or together form a 3- to 7-membered carbocyclus,
   which carbocyclus optionally has in addition to carbon atoms one or two ring members selected from the group consisting of —N(R¹²)—, —N═N—, —C(═O)—, —O— and —S—, and
   which carbocyclus is optionally substituted with one to four substituents selected from R¹⁴;
n 1 to 3;
Q, W, X, Y¹, Y² independently of one another O or S;
Z CH or N;
including their agriculturally acceptable salts, amides, esters or thioesters, provided the compounds of formula (I) have a carboxyl group.

The present invention further discloses phenyluracils of formula (I)

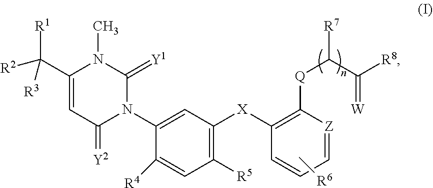

wherein the substituents have the following meanings:
R¹ C₁-C₄-alkyl, C₂-C₄-alkenyl or C₂-C₄-alkynyl;
R² hydrogen, halogen, C₁-C₄-alkyl, C₁-C₄-haloalkyl, C₁-C₄-alkoxy or C₁-C₄-haloalkoxy;
R³ hydrogen, halogen, C₁-C₄-alkyl, C₁-C₄-haloalkyl, C₁-C₄-alkoxy or C₁-C₄-haloalkoxy;
R⁴ H or halogen;
R⁵ halogen, CN, NH₂, NO₂;
R⁶ H, halogen, C₁-C₃-alkyl, C₁-C₃-alkoxy;
R⁷ H, C₁-C₃-alkyl, C₁-C₃-alkoxy;
R⁸ OR⁹, SR⁹, NR¹⁰R¹¹, NR⁹OR⁹, NR⁹S(O)₂R¹⁰ or NR⁹S(O)₂NR¹⁰R¹¹, wherein
R⁹ is hydrogen, C₁-C₆-alkyl, C₃-C₆-alkenyl, C₃-C₆-alkynyl, C₁-C₆-haloalkyl, C₃-C₆-haloalkenyl, C₃-C₆-haloalkynyl, C₁-C₆-cyanoalkyl, C₁-C₆-alkoxy-C₁-C₆-alkyl, C₁-C₆-alkoxy-C₁-C₆-alkoxy-C₁-C₆-alkyl, di(C₁-C₆-alkoxy)C₁-C₆-alkyl, C₁-C₆-haloalkoxy-C₁-C₆-alkyl, C₃-C₆-alkenyloxy-C₁-C₆-alkyl, C₃-C₆-haloalkenyloxy-C₁-C₆-alkyl, C₃-C₆-alkenyloxy-C₁-C₆-alkoxy-C₁-C₆-alkyl, C₁-C₆-alkylthio-C₁-C₆-alkyl, C₁-C₆-alkylsulfinyl-C₁-C₆-alkyl, C₁-C₆-alkylsulfonyl-C₁-C₆-alkyl, C₁-C₆-alkylcarbonyl-C₁-C₆-alkyl, C₁-C₆-alkoxycarbonyl-C₁-C₆-alkyl, C₁-C₆-haloalkoxycarbonyl-C₁-C₆-alkyl, C₃-C₆-alkenyloxycarbonyl-C₁-C₆-alkyl, C₃-C₆-alkynyloxycarbonyl-C₁-C₆-alkyl, amino, (C₁-C₆-alkyl)amino, di(C₁-C₆-alkyl)amino, (C₁-C₆-alkylcarbonyl)amino, amino-C₁-C₆-alkyl, (C₁-C₆-alkyl)amino-C₁-C₆-alkyl, di(C₁-C₆-alkyl)amino-C₁-C₆-alkyl, aminocarbonyl-C₁-C₆-alkyl, (C₁-C₆-alkyl)aminocarbonyl-C₁-C₆-alkyl, di(C₁-C₆-alkyl)aminocarbonyl-C₁-C₆-alkyl,
   —N═CR¹²R¹³, wherein R¹² and R¹³ independently of one another are H, C₁-C₄-alkyl or phenyl;
   C₃-C₆-cycloalkyl, C₃-C₆-cycloalkyl-C₁-C₆-alkyl, C₃-C₆-heterocyclyl, C₃-C₆-heterocyclyl-C₁-C₆-alkyl, phenyl, phenyl-C₁-C₄-alkyl or a 5- or 6 membered heteroaryl,
      wherein each cycloalkyl, heterocyclyl, phenyl or heteroaryl ring can be substituted by one to four substituents selected from R¹⁴ or a 3- to 7-membered carbocyclus,
         which carbocyclus optionally has in addition to carbon atoms one or two ring members selected from the group consisting of —N(R¹²)—, —N═N—, —C(═O)—, —O— and —S—, and
         which carbocyclus is optionally substituted with one to four substituents selected from R¹⁴;
         wherein R¹⁴ is halogen, NO₂, CN, C₁-C₄-alkyl, C₁-C₄-haloalkyl, C₁-C₄-alkoxy or C₁-C₄-alkoxycarbonyl;
R¹⁰, R¹¹ independently of one another are R⁹, or together form a 3- to 7-membered carbocyclus,
   which carbocyclus optionally has in addition to carbon atoms one or two ring members selected from the group consisting of —N(R¹²)—, —N═N—, —C(═O)—, —O— and —S—, and
   which carbocyclus is optionally substituted with one to four substituents selected from R¹⁴;
n 1 to 3;
Q, W, X, Y¹, Y² independently of one another O or S;
Z CH or N;
including their agriculturally acceptable salts, amides, esters or thioesters, provided the compounds of formula (I) have a carboxyl group.

The present invention also provides formulations comprising at least one phenyluracil of formula (I) and auxiliaries customary for formulating crop protection agents.

The present invention also provides the use of phenyluracil of formula (I) as herbicides, i.e. for controlling undesired vegetation.

The present invention furthermore provides a method for controlling undesired vegetation where a herbicidal effective amount of at least one phenyluracil of the formula (I) is allowed to act on plants, their seeds and/or their habitat.

Moreover, the invention relates to processes and intermediates for preparing phenyluracil of formula (I).

If the phenyluracil of formula (I) as described herein are capable of forming geometrical isomers, for example E/Z isomers, it is possible to use both, the pure isomers and mixtures thereof, according to the invention.

If the phenyluracil of formula (I) as described herein have one or more centres of chirality and, as a consequence, are present as enantiomers or diastereomers, it is possible to use both, the pure enantiomers and diastereomers and their mixtures, according to the invention.

If the phenyluracil of formula (I) as described herein have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds.

Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diethylammonium, diisopropylammonium, trimethylammonium, triethylammonium, tris(isopropyl)ammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore phosphonium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine and diethylenetriamine.

Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

Phenyluracil of formula (I) as described herein having a carboxyl group can be employed in the form of the acid, in the form of an agriculturally suitable salt as mentioned above or else in the form of an agriculturally acceptable derivative, for example as amides, such as mono- and di-C₁-C₆-alkylamides or arylamides, as esters, for example as allyl esters, propargyl esters, C₁-C₁₀-alkyl esters, alkoxyalkyl esters, tefuryl ((tetrahydrofuran-2-yl)methyl) esters and also as thioesters, for example as C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, for example the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthio ester.

The organic moieties mentioned in the definition of the variables R¹ to R¹³, are—like the term halogen—collective terms for individual enumerations of the individual group members. The term halogen denotes in each case fluorine, chlorine, bromine or iodine. All hydrocarbon chains can be straight-chain or branched, the prefix Cn-Cm denoting in each case the possible number of carbon atoms in the group.

Examples of such meanings are:
C₁-C₃-alkyl: for example CH₃, C₂H₅, n-propyl and CH(CH₃)₂;
C₁-C₄-alkyl and also the C₁-C₄-alkyl moieties of phenyl-C₁-C₄-alkyl: for example CH₃, C₂H₅, n-propyl, CH(CH₃)₂, n-butyl, CH(CH₃)—C₂H₅, CH₂—CH(CH₃)₂ and C(CH₃)₃;
C₁-C₆-alkyl and also the C₁-C₆-alkyl moieties of C₁-C₆-cyanoalkyl, C₁-C₆-alkyoxy-C₁-C₆-alkyl, C₁-C₆-alkoxy-C₁-C₆-alkoxy-C₁-C₆-alkyl, di(C₁-C₆-alkoxy)C₁-C₆-alkyl, C₁-C₆-haloalkoxy-C₁-C₆-alkyl, C₃-C₆-alkenyloxy-C₁-C₆-alkyl, C₃-C₆-haloalkenyloxy-C₁-C₆-alkyl, C₃-C₆-alkenyloxy-C₁-C₆-alkoxy-C₁-C₆-alkyl, C₁-C₆-alkylthio-C₁-C₆-alkyl, C₁-C₆-alkylsulfinyl-C₁-C₆-alkyl, C₁-C₆-alkylsulfonyl-C₁-C₆-alkyl, C₁-C₆-alkylcarbonyl-C₁-C₆-alkyl, C₁-C₆-alkoxycarbonyl-C₁-C₆-alkyl, C₁-C₆-haloalkoxycarbonyl-C₁-C₆-alkyl, C₃-C₆-alkenyloxycarbonyl-C₁-C₆-alkyl, C₃-C₆-alkynyloxycarbonyl-C₁-C₆-alkyl, (C₁-C₆-alkylcarbonyl)amino, amino-C₁-C₆-alkyl, (C₁-C₆-alkyl)amino-C₁-C₆-alkyl, di(C₁-C₆-alkyl)amino-C₁-C₆-alkyl, aminocarbonyl-C₁-C₆-alkyl, (C₁-C₆-alkyl)aminocarbonyl-C₁-C₆-alkyl, di(C₁-C₆-alkyl)aminocarbonyl-C₁-C₆-alkyl, C₃-C₆-cycloalkyl-C₁-C₆-alkyl, C₃-C₆-heterocyclyl-C₁-C₆-alkyl: C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl or n-hexyl;
C₁-C₄-haloalkyl: C₁-C₄-alkyl as mentioned above which is partially or fully substituted by fluorine, chlorine, bromine and/or iodine, for example, chloromethyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 1,1,2,2,-tetrafluoroethyl and 1-trifluoromethyl-1,2,2,2-tetrafluoroethyl;
C₁-C₆-haloalkyl: C₁-C₄-haloalkyl as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and dodecafluorohexyl;
C₃-C₆-alkenyl and also the C₃-C₆-alkenyl moieties of C₃-C₆-alkenyloxy-C₁-C₆-alkyl, C₃-C₆-alkenyloxy-C₁-C₆-alkoxy-C₁-C₆-alkyl, C₃-C₆-alkenyloxycarbonyl-C₁-C₆-alkyl: for example 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
C₂-C₄-alkenyl: for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl,
C₃-C₆-haloalkenyl and also the C₃-C₆-haloalkenyl moieties of C₃-C₆-haloalkenyloxy-C₁-C₆-alkyl: a C₃-C₆-alkenyl radical as mentioned above which is partially or fully substituted by fluorine, chlorine, bromine and/or iodine, for example 2-chloroprop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl or 2,3-dibromobut-2-en-1-yl;
C₂-C₄-alkynyl: for example ethynyl, 1-propynyl, 2-propynyl, 1-butynyl, 2-butynyl, 3-butynyl, 1-methyl-2-propynyl;
C₃-C₆-alkynyl and also the C₃-C₆-alkynyl moieties of C₃-C₆-alkynyloxycarbonyl-C₁-C₆-alkyl: for example 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
C₃-C₆-haloalkynyl: a C₃-C₆-alkynyl radical as mentioned above which is partially or fully substituted by fluorine, chlorine, bromine and/or iodine, for example 1,1-difluoroprop-2-yn-1-yl, 3-chloroprop-2-yn-1-yl, 3-bromoprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
C₁-C₃-alkoxy: for example methoxy, ethoxy, propoxy;
C₁-C₄-alkoxy and also the C₁-C₄-alkoxy moieties of C₁-C₄-alkyoxycarbonyl: for example methoxy, ethoxy, propoxy, 1-methylethoxy butoxy, 1-methylpropoxy, 2-methylpropoxy and 1,1-dimethylethoxy;
C₁-C₆-alkoxy and also the C₁-C₆-alkoxy moieties of C₁-C₆-alkyoxy-C₁-C₆-alkyl, C₁-C₆-alkoxy-C₁-C₆-alkoxy-C₁-C₆-alkyl, di(C₁-C₆-alkoxy)C₁-C₆-alkyl, C₃-C₆-alkenyloxy-C₁-C₆-alkoxy-C₁-C₆-alkyl, C₁-C₆-alkoxycarbonyl-C₁-C₆-alkyl: C₁-C₄-alkoxy as mentioned above, and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy.
C₁-C₄-haloalkoxy: a C₁-C₄-alkoxy radical as mentioned above which is partially or fully substituted by fluorine, chlorine, bromine and/or iodine, i.e., for example, fluoromethoxy, difluoromethoxy, trifluoromethoxy, chlorodifluoromethoxy, bromodifluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy and nonafluorobutoxy;
C₁-C₆-haloalkoxy and also the C₁-C₆-haloalkoxy moieties of C₁-C₆-haloalkoxy-C₁-C₆-alkyl, C₁-C₆-haloalkoxycarbonyl-C₁-C₆-alkyl: a C₁-C₄-haloalkoxy as mentioned above, and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy and dodecafluorohexoxy;
C₁-C₄-alkylthio: for example methylthio, ethylthio, propylthio, 1-methylethylthio, butylthio, 1-methylpropylthio, 2-methylpropylthio and 1,1-dimethylethylthio;
C₁-C₆-alkylthio and also the C₁-C₆-alkylthio moieties of C₁-C₆-alkylthio-C₁-C₆-alkyl: C₁-C₄-alkylthio as mentioned above, and also, for example, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;
C₁-C₆-alkylsulfinyl (C₁-C₆-alkyl-S(═O)—) and also the C₁-C₆-alkylsulfinyl moieties of C₁-C₆-alkylsulfinyl-C₁-C₆-alkyl: for example methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropylsulfinyl, hexylsulfinyl, 1-methylpentylsulfinyl, 2-methylpentylsulfinyl, 3-methylpentylsulfinyl, 4-methylpentyl-sulfinyl, 1,1-dimethylbutylsulfinyl, 1,2-dimethylbutylsulfinyl, 1,3-dimethylbutyl-sulfinyl, 2,2-dimethylbutylsulfinyl, 2,3-dimethylbutylsulfinyl, 3,3-dimethylbutyl-sulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl;
C₁-C₆-alkylsulfonyl (C₁-C₆-alkyl-S(O)₂—) and also the C₁-C₆-alkylsulfonyl moieties of C₁-C₆-alkylsulfonyl-C₁-C₆-alkyl: for example methylsulfonyl, ethylsulfonyl, propylsulfonyl, 1-methylethylsulfonyl, butylsulfonyl, 1-methylpropylsulfonyl, 2-methyl-propylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethyl-propylsulfonyl, 1,2,2-trimethylpropylsulfonyl, 1-ethyl-1-methylpropylsulfonyl and 1-ethyl-2-methylpropylsulfonyl;
(C₁-C₄-alkyl)amino: for example methylamino, ethylamino, propylamino, 1-methylethylamino, butylamino, 1-methylpropylamino, 2-methylpropylamino or 1,1-dimethylethylamino;
(C₁-C₆-alkyl)amino and also the (C₁-C₆-alkyl)amino moiety in (C₁-C₆-alkyl)amino-C₁-C₆-alkyl: (C₁-C₄-alkyl)amino as mentioned above, and also, for example, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutyl-amino 3,3-dimethylbutylamino, 1-ethylbutylamino, 2-ethylbutylamino, 1,1,2-trimethylpropylamino, 1,2,2-trimethyl-propylamino, 1-ethyl-1-methylpropylamino or 1-ethyl-2-methylpropylamino;
di(C₁-C₄-alkyl)amino: for example N,N-dimethylamino, N,N-diethylamino, N,N-di(1-methylethyl)amino, N,N-dipropylamino, N,N-dibutylamino, N,N-di(1-methylpropyl)amino, N,N-di(2-methylpropyl)amino, N,N-di(1,1-dimethylethyl)amino, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino or N-(1,1-dimethylethyl)-N-(2-methylpropyl)amino;
di(C₁-C₆-alkyl)amino and also the di(C₁-C₆-alkyl)amino moiety of di(C₁-C₆-alkyl)amino-C₁-C₆-alkyl: di(C₁-C₄-alkyl)amino as mentioned above, and also, for example, N-methyl-N-pentylamino, N-methyl-N-(1-methylbutyl)amino, N-methyl-N-(2-methylbutyl)amino, N-methyl-N-(3-methylbutyl)amino, N-methyl-N-(2,2-dimethylpropyl)amino, N-methyl-N-(1-ethylpropyl)amino, N-methyl-N-hexylamino, N-methyl-N-(1,1-dimethylpropyl)amino, N-methyl-N-(1,2-dimethylpropyl)amino, N-methyl-N-(1-methylpentyl)amino, N-methyl-N-(2-methylpentyl)amino, N-methyl-N-(3-methylpentyl)amino, N-methyl-N-(4-methylpentyl)amino, N-methyl-N-(1,1-dimethylbutyl)amino, N-methyl-N-(1,2-dimethylbutyl)amino, N-methyl-N-(1,3-dimethylbutyl)amino, N-methyl-N-(2,2-dimethylbutyl)amino, N-methyl-N-(2,3-dimethylbutyl)amino, N-methyl-N-(3,3-dimethylbutyl)amino, N-methyl-N-(1-ethylbutyl)amino, N-methyl-N-(2-ethylbutyl)amino, N-methyl-N-(1,1,2-trimethylpropyl)amino, N-methyl-N-(1,2,2-trimethylpropyl)amino, N-methyl-N-(1-ethyl-1-methylpropyl)amino, N-methyl-N-(1-ethyl-2-methylpropyl)amino, N-ethyl-N-pentylamino, N-ethyl-N-(1-methylbutyl)amino, N-ethyl-N-(2-methylbutyl)amino, N-ethyl-N-(3-methylbutyl)amino, N-ethyl-N-(2,2-dimethylpropyl)amino, N-ethyl-N-(1-ethylpropyl)amino, N-ethyl-N-hexylamino, N-ethyl-N-(1,1-dimethylpropyl)amino, N-ethyl-N-(1,2-dimethylpropyl)amino, N-ethyl-N-(1-methylpentyl)amino, N-ethyl-N-(2-methylpentyl)amino, N-ethyl-N-(3-methylpentyl)amino, N-ethyl-N-(4-methylpentyl)amino, N-ethyl-N-(1,1-dimethylbutyl)amino, N-ethyl-N-(1,2-dimethylbutyl)amino, N-ethyl-N-(1,3-dimethylbutyl)amino, N-ethyl-N-(2,2-dimethylbutyl)amino, N-ethyl-N-(2,3-dimethylbutyl)amino, N-ethyl-N-(3,3-dimethylbutyl)amino, N-ethyl-N-(1-ethylbutyl)amino, N-ethyl-N-(2-ethylbutyl)amino, N-ethyl-N-(1,1,2-trimethylpropyl)amino, N-ethyl-N-(1,2,2-trimethylpropyl)amino, N-ethyl-N-(1-ethyl-1-methylpropyl)amino, N-ethyl-N-(1-ethyl-2-methylpropyl)amino, N-propyl-N-pentylamino, N-butyl-N-pentylamino, N,N-dipentylamino, N-propyl-N-hexylamino, N-butyl-N-hexylamino, N-pentyl-N-hexylamino or N,N-dihexylamino;
C₃-C₆-cycloalkyl and also the cycloalkyl moieties of C₃-C₆-cycloalkyl-C₁-C₆-alkyl: monocyclic saturated hydrocarbons having 3 to 6 ring members, such as cyclopropyl, cyclobutyl, cyclopentyl and cyclohexyl;
C₃-C₆-heterocyclyl and also the heterocyclyl moieties of C₃-C₆-heterocyclyl-C₁-C₆-alkyl: aliphatic heterocycle having 3 to 6 ring members which, in addition to carbon atoms, contains 1 to 4 nitrogen atoms, or 1 to 3 nitrogen atoms and an oxygen or sulphur atom, or an oxygen or a sulphur atom, for example
   three- or four-membered heterocycles like 2-oxetanyl, 3-oxetanyl, 2-thietanyl, 3-thietanyl, 1-azetidinyl, 2-azetidinyl, 1-azetinyl, 2-azetinyl; five-membered saturated heterocycles like 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 2-isothiazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 3-oxazolidinyl, 1,2,4-oxadiazolidin-3-yl, 1,2,4-oxadiazolidin-5-yl, 3-thiazolidinyl, 1,2,4-thiadiazolidin-3-yl, 1,2,4-thiadiazolidin-5-yl, 1,2,4-triazolidin-3-yl, 1,2,4-oxadiazolidin-2-yl, 1,2,4-oxadiazolidin-4-yl, 1,3,4-oxadiazolidin-2-yl, 1,2,4-thiadiazolidin-2-yl, 1,2,4-thiadiazolidin-4-yl, 1,3,4-thiadiazolidin-2-yl, 1,2,4-triazolidin-1-yl, 1,3,4-triazolidin-2-yl; six-membered saturated heterocycles like 1-piperidinyl, 2-piperidinyl, 3-piperidinyl, 4-piperidinyl, 1,3-dioxan-5-yl, 1,4-dioxanyl, 1,3-dithian-5-yl, 1,3-dithianyl, 1,3-oxathian-5-yl, 1,4-oxathianyl, 2-tetrahydropyranyl, 3-tetrahydopyranyl, 4-tetrahydropyranyl, 2-tetrahydrothiopyranyl, 3-tetrahydrothiopyranyl, 4-tetrahydrothiopyranyl, 1-hexahydropyridazinyl, 3-hexahydropyridazinyl, 4-hexahydropyridazinyl, 1-hexahydropyrimidinyl, 2-hexahydropyrimidinyl, 4-hexahydropyrimidinyl, 5-hexahydropyrimidinyl, 1-piperazinyl, 2-piperazinyl, 1,3,5-hexahydrotriazin-1-yl, 1,3,5-hexahydrotriazin-2-yl, 1,2,4-hexahydrotriazin-1-yl, 1,2,4-hexahydrotriazin-3-yl, tetrahydro-1,3-oxazin-1-yl, tetrahydro-1,3-oxazin-2-yl, tetrahydro-1,3-oxazin-6-yl, 1-morpholinyl, 2-morpholinyl, 3-morpholinyl;
5- or 6 membered heteroaryl: aromatic heteroaryl having 5 or 6 ring members which, in addition to carbon atoms, contains 1 to 4 nitrogen atoms, or 1 to 3 nitrogen atoms and an oxygen or sulphur atom, or an oxygen or a sulphur atom, for example 5-membered aromatic rings like furyl (for example 2-furyl, 3-furyl), thienyl (for example 2-thienyl, 3-thienyl), pyrrolyl (for example pyrrol-2-yl, pyrrol-3-yl), pyrazolyl (for example pyrazol-3-yl, pyrazol-4-yl), isoxazolyl (for example isoxazol-3-yl, isoxazol-4-yl, isoxazol-5-yl), isothiazolyl (for example isothiazol-3-yl, isothiazol-4-yl, isothiazol-5-yl), imidazolyl (for example imidazole-2-yl, imidazole-4-yl), oxazolyl (for example oxazol-2-yl, oxazol-4-yl, oxazol-5-yl), thiazolyl (for example thiazol-2-yl, thiazol-4-yl, thiazol-5-yl), oxadiazolyl (for example 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl), thiadiazolyl (for example 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl), triazolyl (for example 1,2,3-triazol-4-yl, 1,2,4-triazol-3-yl); 1-tetrazolyl; 6-membered aromatic rings like pyridyl (for example pyridine-2-yl, pyridine-3-yl, pyridine-4-yl), pyrazinyl (for example pyridazin-3-yl, pyridazin-4-yl), pyrimidinyl (for example pyrimidin-2-yl, pyrimidin-4-yl, pyrimidin-5-yl), pyrazin-2-yl, triazinyl (for example 1,3,5-triazin-2-yl, 1,2,4-triazin-3-yl, 1,2,4-triazin-5-yl, 1,2,4-triazin-6-yl);
3- to 7-membered carbocyclus: a three- to seven-membered monocyclic, saturated, partial unsaturated or aromatic cycle having three to seven ring members which comprises apart from carbon atoms optionally one or two ring members selected from the group consisting of —N(R¹²), —N═N—, —C(═O)—, —O— and —S—.

The preferred embodiments of the invention mentioned herein below have to be understood as being preferred either independently from each other or in combination with one another.

According to a preferred embodiment of the invention preference is also given to those phenyluracils of formula (I), wherein the variables, either independently of one another or in combination with one another, have the following meanings:

Preferred are the phenyluracils of formula (I) wherein
R¹ is C₁-C₄-alkyl;
   preferably is CH₃.

Also preferred are the phenyluracils of formula (I) wherein
R² is H, halogen or C₁-C₄-alkyl;
   preferably is H, F, Cl, or CH₃;
   more preferred is H or F;
   particularly preferred is H;
   also particularly preferred is F.

Also preferred are the phenyluracils of formula (I) wherein
R² is H or halogen;
   preferably is halogen;
   more preferably is F.

Also preferred are the phenyluracils of formula (I) wherein
R³ is H, halogen or C₁-C₄-alkyl;
   preferably is H, F, Cl or CH₃;
   more preferred is H or F;
   particularly preferred is H;
   also particularly preferred is F.

Also preferred are the phenyluracils of formula (I) wherein
R³ is halogen;
preferably is F.

Also preferred are the phenyluracils of formula (I) wherein
R⁴ is H, F or Cl;
   particularly preferred is H or F;
   especially preferred is H;
   also particularly preferred is H or Cl;
   especially preferred is Cl;
   also particularly preferred is F or Cl;
   especially preferred is F.

Also preferred are the phenyluracils of formula (I) wherein
R⁴ is halogen;
   preferably is F.

Also preferred are the phenyluracils of formula (I) wherein
R⁵ is halogen or CN;
   preferably F, C₁, Br or CN;
   particularly preferred is Cl, Br or CN;
especially preferred is Cl or Br;
   more preferred is Cl;
   also more preferred is Br;

Also preferred are the phenyluracils of formula (I) wherein
R⁵ is halogen;
   preferably is C₁.

Also preferred are the phenyluracils of formula (I) wherein
R⁶ is H, F, C₁-C₃-alkyl or C₁-C₃-alkoxy;
   particularly preferred is H, CH₃ or OCH₃,
   especially preferred is H.

Also preferred are the phenyluracils of formula (I) wherein
R⁷ is H, CH₃ or OCH₃;
   particularly preferred is H or OCH₃;
   especially preferred is H;
   also especially preferred is OCH₃.

Also preferred are the phenyluracils of formula (I) wherein
R⁷ is H or C₁-C₃-alkoxy;
   preferably is C₁-C₃-alkoxy.

Also preferred are the phenyluracils of formula (I) wherein
R⁸ is OR⁹, SR⁹, NR¹⁰R¹¹, NR⁹S(O)₂R¹⁰ or NR⁹S(O)₂NR¹⁰R¹¹;
   particularly preferred is OR⁹, NR¹⁰R¹¹, NR⁹S(O)₂R¹⁰ or NR⁹S(O)₂NR¹⁰R¹¹;
   especially preferred OR⁹, NR⁹S(O)₂R¹⁰ or NR⁹S(O)₂NR¹⁰R¹¹;
   especially preferred is OR⁹ or NR⁹S(O)₂R¹⁰;
   more preferred is OR⁹.

Also preferred are the phenyluracils of formula (I) wherein
R⁹ is hydrogen, C₁-C₆-alkyl, C₃-C₆-alkenyl, C₃-C₆-alkynyl, C₁-C₆-haloalkyl, C₃-C₆-haloalkenyl, C₃-C₆-haloalkynyl, C₁-C₆-cyanoalkyl, C₁-C₆-alkoxy-C₁-C₆-alkyl, C₁-C₆-alkoxy-C₁-C₆-alkoxy-C₁-C₆-alkyl, di(C₁-C₆-alkoxy)C₁-C₆-alkyl, C₁-C₆-haloalkoxy-C₁-C₆-alkyl, C₃-C₆-alkenyloxy-C₁-C₆-alkyl, C₃-C₆-haloalkenyloxy-C₁-C₆-alkyl, C₃-C₆-alkenyloxy-C₁-C₆-alkoxy-C₁-C₆-alkyl, C₁-C₆-alkylthio-C₁-C₆-alkyl, C₁-C₆-alkylsulfinyl-C₁-C₆-alkyl, C₁-C₆-alkylsulfonyl-C₁-C₆-alkyl, C₁-C₆-alkylcarbonyl-C₁-C₆-alkyl, C₁-C₆-alkoxycarbonyl-C₁-C₆-alkyl, C₁-C₆-haloalkoxycarbonyl-C₁-C₆-alkyl, C₃-C₆-alkenyloxycarbonyl-C₁-C₆-alkyl, C₃-C₆-alkynyloxycarbonyl-C₁-C₆-alkyl, amino, (C₁-C₆-alkyl)amino, di(C₁-C₆-alkyl)amino, (C₁-C₆-alkylcarbonyl)amino, amino-C₁-C₆-alkyl, (C₁-C₆-alkyl)amino-C₁-C₆-alkyl, di(C₁-C₆-alkyl)amino-C₁-C₆-alkyl, aminocarbonyl-C₁-C₆-alkyl, (C₁-C₆-alkyl)aminocarbonyl-C₁-C₆-alkyl, di(C₁-C₆-alkyl)aminocarbonyl-C₁-C₆-alkyl, —N═CR¹²R¹³
   wherein R¹² and R¹³ independently of one another are H, C₁-C₄-alkyl or phenyl; C₃-C₆-cycloalkyl, C₃-C₆-cycloalkyl-C₁-C₆-alkyl, C₃-C₆-heterocyclyl, phenyl, phenyl-C₁-C₄-alkyl or a 5- or 6 membered heteroaryl,
   wherein each cycloalkyl, heterocyclyl, phenyl or heteroaryl ring can be substituted by one to four substituents selected from R¹⁴ or a 3- to 7-membered carbocyclus,
      which carbocyclus optionally has in addition to carbon atoms one or two ring members selected from the group consisting of —N(R¹²)—, —N═N—, —C(═O)—, —O— and —S—, and
      which carbocyclus is optionally substituted with one to four substituents selected from R¹⁴,
         wherein R¹⁴ is halogen, NO₂, CN, C₁-C₄-alkyl, C₁-C₄-haloalkyl, C₁-C₄-alkoxy or C₁-C₄-alkoxycarbonyl;
   preferably is hydrogen, C₁-C₆-alkyl, C₃-C₆-alkenyl, C₃-C₆-alkynyl or C₁-C₆-haloalkyl;
   particularly preferred is hydrogen, C₁-C₆-alkyl, C₃-C₆-alkenyl or C₃-C₆-alkynyl;
   especially preferred is hydrogen or C₁-C₄-alkyl;
   more preferred is hydrogen, CH₃ or C₂H₅.

Also preferred are the phenyluracils of formula (I) wherein
R⁹ is H, C₁-C₆-alkyl, C₁-C₆-cyanoalkyl, C₁-C₆-alkoxy-C₁-C₆-alkyl or C₃-C₆-heterocyclyl-C₁-C₆-alkyl;
   preferably is H, CH₃, C2H5, CH₂CN, C₂H₄OCH₃ or CH₂-2-tetrahydrofuranyl.

Also preferred are the phenyluracils of formula (I) wherein
R⁹ is hydrogen, C₁-C₆-alkyl or C₁-C₆-alkoxy-C₁-C₆-alkyl;
   preferably is C₁-C₆-alkyl or C₁-C₆-alkoxy-C₁-C₆-alkyl;
   particularly preferred is C₁-C₆-alkyl.

Also preferred are the phenyluracils of formula (I) wherein
R¹⁰ is C₁-C₆-alkyl or C₃-C₆-alkoxy;
   particularly preferred is C₁-C₆-alkyl;
   more preferred is CH₃.

Also preferred are the phenyluracils of formula (I) wherein
R¹¹ is H, C₁-C₆-alkyl or C₁-C₆-alkoxycarbonyl-C₁-C₆-alkyl;
   particularly preferred is H or C₁-C₆-alkyl;
   more preferred is H;
   also more preferred is C₁-C₆-alkyl.

Also preferred are the phenyluracils of formula (I) wherein
R¹² is phenyl or C₁-C₄-alkyl;
   particularly preferred is phenyl or CH₃;
   also particularly preferred is phenyl;
   also particularly preferred is C₁-C₄-alkyl.

Also preferred are the phenyluracils of formula (I) wherein
R¹³ is phenyl or C₁-C₄-alkyl;
   particularly preferred is phenyl or CH₃;
   also particularly preferred is phenyl;
   also particularly preferred is C₁-C₄-alkyl.

Also preferred are the phenyluracils of formula (I) wherein
R¹⁴ is halogen or C₁-C₆-alkyl;
   particularly preferred is F, Cl or CH₃;
   also particularly preferred is halogen;
   especially preferred is F or Cl;
   also particularly preferred is C₁-C₆-alkyl;
   especially preferred is CH₃.

Also preferred are the phenyluracils of formula (I) wherein
n is 1 or 2;
   particularly preferred is 2;
   also particularly preferred is 1.

Also preferred are the phenyluracils of formula (I) wherein
Q is O,
   also preferably is S.

Also preferred are the phenyluracils of formula (I) wherein
W is O,
   also preferably is S.

Also preferred are the phenyluracils of formula (I) wherein
X is O,
   also preferably is S.

Also preferred are the phenyluracils of formula (I) wherein
Y¹ is O,
   also preferably is S.

Also preferred are the phenyluracils of formula (I) wherein
Y² is O,
   also preferably is S.

Also preferred are the phenyluracils of formula (I) wherein
Z is CH,
   also preferably is N.

Also preferred are the phenyluracils of formula (I) wherein
R¹ is CH₃;
R² is F;
R³ is F;
R⁴ is F
R⁵ is Cl or Br;
R⁶ is H;
R⁷ is H or OCH₃;
R⁸ is OR⁹, wherein R⁹ is H, CH³ or C₂H₅;
n is 1
Q, W, X, Y¹ and Y² are O.

Also preferred are the phenyluracils of formula (I) wherein
R¹ is CH₃;
R² is F;
R³ is F;
R⁴ is F;
R⁵ is Cl or Br;
R⁸ is H;
R⁷ is H;
R⁸ is OR⁹, wherein R⁹ is H, CH³ or C₂H₅;
n is 1
Q, W, X, Y¹ and Y² are O.

Also preferred are the phenyluracils of formula (I) wherein
R¹ is CH₃;
R² is F;
R³ is F;
R⁴ is F;
R⁵ is Cl;
R⁸ is H;
R⁷ is H;
R⁸ is OR⁹,
   wherein R⁹ is hydrogen, C₁-C₆-alkyl or C₁-C₆-alkoxy-C₁-C₆-alkyl;
      preferably is C₁-C₆-alkyl or C₁-C₆-alkoxy-C₁-C₆-alkyl;
      particularly preferred is C₁-C₆-alkyl;
n is 1
Q, W, X, Y¹ and Y² are O.

Also preferred are the phenyluracils of formula (I) wherein
R¹ is CH₃;
R² is F;
R³ is F;
R⁴ is F;
R⁵ is Cl or Br;
R⁸ is H;
R⁷ is OCH₃;
R⁸ is OR⁹,
   wherein R⁹ is H, CH³ or C₂H₅;
n is 1
Q, W, X, Y¹ and Y² are O.

Also preferred are the phenyluracils of formula (I) wherein
R¹ is CH₃, C₂-C₄-alkenyl or C₂-C₄-alkynyl;
R² is H, F, C₁-C₄-alkyl, C₁-C₄-haloalkyl, C₁-C₄-alkoxy or C₁-C₄-haloalkoxy;
R³ is H, F, C₁-C₄-alkyl, C₁-C₄-haloalkyl, C₁-C₄-alkoxy or C₁-C₄-haloalkoxy;
   preferably
R¹ is CH₃;
R² is H, F;
R³ is F;
   particularly preferred
R¹ is CH₃;
R² is F;
R³ is F.

Also preferred are the phenyluracils of formula (I) wherein
R¹ is CH₃, C₂-C₄-alkenyl or C₂-C₄-alkynyl;
R² is H, F, C₁-C₄-alkyl, C₁-C₄-haloalkyl, C₁-C₄-alkoxy or C₁-C₄-haloalkoxy;
R³ is H, F, C₁-C₄-alkyl, C₁-C₄-haloalkyl, C₁-C₄-alkoxy or C₁-C₄-haloalkoxy;
Z is CH;
   preferably
R¹ is CH₃;
R² is H, F;
R³ is F;
Z is CH.

Also preferred are the phenyluracils of formula (I) wherein
R¹ is CH₃, C₂-C₄-alkenyl or C₂-C₄-alkynyl;
R² is H, F, C₁-C₄-alkyl, C₁-C₄-haloalkyl, C₁-C₄-alkoxy or C₁-C₄-haloalkoxy;
R³ is H, F, C₁-C₄-alkyl, C₁-C₄-haloalkyl, C₁-C₄-alkoxy or C₁-C₄-haloalkoxy;
Z is N;
   preferably
R¹ is CH₃;
R² is H, F;
R³ is F;
Z is N.

Preferred are the phenyluracils of formula (I.1) (corresponds to formula (I) wherein R¹ is CH₃, R³ and R⁴ are F, R⁶ is H, n is 1, Q, W, X, Y¹ and Y² are O),

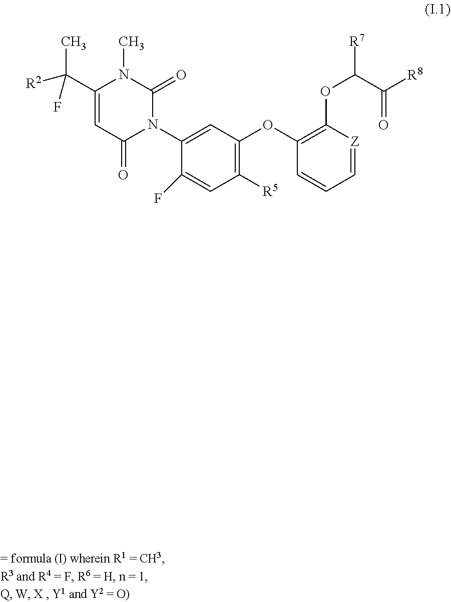

wherein
R² is H or halogen,
R⁵ is halogen;
R⁷ is H or C₁-C₃-alkoxy;
R⁸ is OR⁹, wherein
R⁹ is H, C₁-C₆-alkyl, C₁-C₆-cyanoalkyl, C₁-C₆-alkoxy-C₁-C₆-alkyl or C₃-C₆-heterocyclyl-C₁-C₆-alkyl;
Z is CH or N;

Especially preferred are the phenyluracils of formula (I.1), wherein
R² is H or F,
R⁵ is Cl or Br;
R⁷ is H or OCH₃;
R⁸ is OR⁹, wherein
R⁹ is H, CH₃, C2H5, CH₂CN, C₂H₄OCH₃ or CH₂-2-tetrahydrofuranyl;
Z is CH or N.

Particular preference is given to phenyluracils of formula (I.a) (corresponds to formula (I) wherein R¹ is CH₃, R³ and R⁴ are F, R⁶ is H, n is 1, Q, W, X, Y¹ and Y² are O and Z is CH),

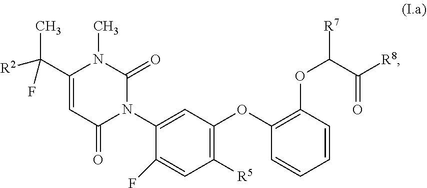

wherein the variables R², R⁵, R⁷ and R⁸ have the meanings, in particular the preferred meanings, as defined above.

Special preference is given to the phenyluracils of the formulae (I.a.1) to (I.a.168), preferably (I.a.1) to (I.a.144), of Table A, where the definitions of the variables R⁴, R⁵, R⁷ and R⁸ are of particular importance for the compounds according to the invention not only in combination with one another but in each case also on their own:

TABLE A

| No. | R² | R⁵ | R⁷ | R⁸ |
|---|---|---|---|---|
| I.a.1. | H | F | H | OH |
| I.a.2. | H | F | H | OCH₃ |
| I.a.3. | H | F | H | OC₂H₅ |
| I.a.4. | H | F | H | OCH(CH₃)₂ |
| I.a.5. | H | F | H | OCH₂CH₂CH₃ |
| I.a.6. | H | F | H | OCH₂CH(CH₃)₂ |
| I.a.7. | H | F | CH₃ | OH |
| I.a.8. | H | F | CH₃ | OCH₃ |
| I.a.9. | H | F | CH₃ | OC₂H₅ |
| I.a.10. | H | F | CH₃ | OCH(CH₃)₂ |
| I.a.11. | H | F | CH₃ | OCH₂CH₂CH₃ |
| I.a.12. | H | F | CH₃ | OCH₂CH(CH₃)₂ |
| I.a.13. | H | F | OCH₃ | OH |
| I.a.14. | H | F | OCH₃ | OCH₃ |
| I.a.15. | H | F | OCH₃ | OC₂H₅ |
| I.a.16. | H | F | OCH₃ | OCH(CH₃)₂ |
| I.a.17. | H | F | OCH₃ | OCH₂CH₂CH₃ |
| I.a.18. | H | F | OCH₃ | OCH₂CH(CH₃)₂ |
| I.a.19. | H | Cl | H | OH |
| I.a.20. | H | Cl | H | OCH₃ |
| I.a.21. | H | Cl | H | OC₂H₅ |
| I.a.22. | H | Cl | H | OCH(CH₃)₂ |
| I.a.23. | H | Cl | H | OCH₂CH₂CH₃ |
| I.a.24. | H | Cl | H | OCH₂CH(CH₃)₂ |
| I.a.25. | H | Cl | CH₃ | OH |
| I.a.26. | H | Cl | CH₃ | OCH₃ |
| I.a.27. | H | Cl | CH₃ | OC₂H₅ |
| I.a.28. | H | Cl | CH₃ | OCH(CH₃)₂ |
| I.a.29. | H | Cl | CH₃ | OCH₂CH₂CH₃ |
| I.a.30. | H | Cl | CH₃ | OCH₂CH(CH₃)₂ |
| I.a.31. | H | Cl | OCH₃ | OH |
| I.a.32. | H | Cl | OCH₃ | OCH₃ |
| I.a.33. | H | Cl | OCH₃ | OC₂H₅ |
| I.a.34. | H | Cl | OCH₃ | OCH(CH₃)₂ |
| I.a.35. | H | Cl | OCH₃ | OCH₂CH₂CH₃ |
| I.a.36. | H | Cl | OCH₃ | OCH₂CH(CH₃)₂ |
| I.a.37. | H | Br | H | OH |
| I.a.38. | H | Br | H | OCH₃ |
| I.a.39. | H | Br | H | OC₂H₅ |
| I.a.40. | H | Br | H | OCH(CH₃)₂ |
| I.a.41. | H | Br | H | OCH₂CH₂CH₃ |
| I.a.42. | H | Br | H | OCH₂CH(CH₃)₂ |
| I.a.43. | H | Br | CH₃ | OH |
| I.a.44. | H | Br | CH₃ | OCH₃ |
| I.a.45. | H | Br | CH₃ | OC₂H₅ |
| I.a.46. | H | Br | CH₃ | OCH(CH₃)₂ |
| I.a.47. | H | Br | CH₃ | OCH₂CH₂CH₃ |
| I.a.48. | H | Br | CH₃ | OCH₂CH(CH₃)₂ |
| I.a.49. | H | Br | OCH₃ | OH |
| I.a.50. | H | Br | OCH₃ | OCH₃ |
| I.a.51. | H | Br | OCH₃ | OC₂H₅ |
| I.a.52. | H | Br | OCH₃ | OCH(CH₃)₂ |
| I.a.53. | H | Br | OCH₃ | OCH₂CH₂CH₃ |
| I.a.54. | H | Br | OCH₃ | OCH₂CH(CH₃)₂ |
| I.a.55. | H | CN | H | OH |
| I.a.56. | H | CN | H | OCH₃ |
| I.a.57. | H | CN | H | OC₂H₅ |
| I.a.58. | H | CN | H | OCH(CH₃)₂ |
| I.a.59. | H | CN | H | OCH₂CH₂CH₃ |
| I.a.60. | H | CN | H | OCH₂CH(CH₃)₂ |
| I.a.61. | H | CN | CH₃ | OH |
| I.a.62. | H | CN | CH₃ | OCH₃ |
| I.a.63. | H | CN | CH₃ | OC₂H₅ |
| I.a.64. | H | CN | CH₃ | OCH(CH₃)₂ |
| I.a.65. | H | CN | CH₃ | OCH₂CH₂CH₃ |
| I.a.66. | H | CN | CH₃ | OCH₂CH(CH₃)₂ |
| I.a.67. | H | CN | OCH₃ | OH |
| I.a.68. | H | CN | OCH₃ | OCH₃ |
| I.a.69. | H | CN | OCH₃ | OC₂H₅ |
| I.a.70. | H | CN | OCH₃ | OCH(CH₃)₂ |
| I.a.71. | H | CN | OCH₃ | OCH₂CH₂CH₃ |
| I.a.72. | H | CN | OCH₃ | OCH₂CH(CH₃)₂ |
| I.a.73. | F | F | H | OH |
| I.a.74. | F | F | H | OCH₃ |
| I.a.75. | F | F | H | OC₂H₅ |
| I.a.76. | F | F | H | OCH(CH₃)₂ |
| I.a.77. | F | F | H | OCH₂CH₂CH₃ |
| I.a.78. | F | F | H | OCH₂CH(CH₃)₂ |
| I.a.79. | F | F | CH₃ | OH |
| I.a.80. | F | F | CH₃ | OCH₃ |
| I.a.81. | F | F | CH₃ | OC₂H₅ |
| I.a.82. | F | F | CH₃ | OCH(CH₃)₂ |
| I.a.83. | F | F | CH₃ | OCH₂CH₂CH₃ |
| I.a.84. | F | F | CH₃ | OCH₂CH(CH₃)₂ |
| I.a.85. | F | F | OCH₃ | OH |
| I.a.86. | F | F | OCH₃ | OCH₃ |
| I.a.87. | F | F | OCH₃ | OC₂H₅ |
| I.a.88. | F | F | OCH₃ | OCH(CH₃)₂ |
| I.a.89. | F | F | OCH₃ | OCH₂CH₂CH₃ |
| I.a.90. | F | F | OCH₃ | OCH₂CH(CH₃)₂ |
| I.a.91. | F | Cl | H | OH |
| I.a.92. | F | Cl | H | OCH₃ |
| I.a.93. | F | Cl | H | OC₂H₅ |
| I.a.94. | F | Cl | H | OCH(CH₃)₂ |
| I.a.95. | F | Cl | H | OCH₂CH₂CH₃ |
| I.a.96. | F | Cl | H | OCH₂CH(CH₃)₂ |
| I.a.97. | F | Cl | CH₃ | OH |
| I.a.98. | F | Cl | CH₃ | OCH₃ |
| I.a.99. | F | Cl | CH₃ | OC₂H₅ |
| I.a.100. | F | Cl | CH₃ | OCH(CH₃)₂ |
| I.a.101. | F | Cl | CH₃ | OCH₂CH₂CH₃ |
| I.a.102. | F | Cl | CH₃ | OCH₂CH(CH₃)₂ |
| I.a.103. | F | Cl | OCH₃ | OH |
| I.a.104. | F | Cl | OCH₃ | OCH₃ |
| I.a.105. | F | Cl | OCH₃ | OC₂H₅ |
| I.a.106. | F | Cl | OCH₃ | OCH(CH₃)₂ |
| I.a.107. | F | Cl | OCH₃ | OCH₂CH₂CH₃ |
| I.a.108. | F | Cl | OCH₃ | OCH₂CH(CH₃)₂ |
| I.a.109. | F | Br | H | OH |
| I.a.110. | F | Br | H | OCH₃ |
| I.a.111. | F | Br | H | OC₂H₅ |
| I.a.112. | F | Br | H | OCH(CH₃)₂ |
| I.a.113. | F | Br | H | OCH₂CH₂CH₃ |
| I.a.114. | F | Br | H | OCH₂CH(CH₃)₂ |
| I.a.115. | F | Br | CH₃ | OH |
| I.a.116. | F | Br | CH₃ | OCH₃ |
| I.a.117. | F | Br | CH₃ | OC₂H₅ |
| I.a.118. | F | Br | CH₃ | OCH(CH₃)₂ |
| I.a.119. | F | Br | CH₃ | OCH₂CH₂CH₃ |
| I.a.120. | F | Br | CH₃ | OCH₂CH(CH₃)₂ |
| I.a.121. | F | Br | OCH₃ | OH |
| I.a.122. | F | Br | OCH₃ | OCH₃ |
| I.a.123. | F | Br | OCH₃ | OC₂H₅ |
| I.a.124. | F | Br | OCH₃ | OCH(CH₃)₂ |
| I.a.125. | F | Br | OCH₃ | OCH₂CH₂CH₃ |
| I.a.126. | F | Br | OCH₃ | OCH₂CH(CH₃)₂ |
| I.a.127. | F | CN | H | OH |
| I.a.128. | F | CN | H | OCH₃ |
| I.a.129. | F | CN | H | OC₂H₅ |
| I.a.130. | F | CN | H | OCH(CH₃)₂ |
| I.a.131. | F | CN | H | OCH₂CH₂CH₃ |
| I.a.132. | F | CN | H | OCH₂CH(CH₃)₂ |
| I.a.133. | F | CN | CH₃ | OH |
| I.a.134. | F | CN | CH₃ | OCH₃ |
| I.a.135. | F | CN | CH₃ | OC₂H₅ |
| I.a.136. | F | CN | CH₃ | OCH(CH₃)₂ |
| I.a.137. | F | CN | CH₃ | OCH₂CH₂CH₃ |
| I.a.138. | F | CN | CH₃ | OCH₂CH(CH₃)₂ |
| I.a.139. | F | CN | OCH₃ | OH |
| I.a.140. | F | CN | OCH₃ | OCH₃ |
| I.a.141. | F | CN | OCH₃ | OC₂H₅ |
| I.a.142. | F | CN | OCH₃ | OCH(CH₃)₂ |
| I.a.143. | F | CN | OCH₃ | OCH₂CH₂CH₃ |
| I.a.144. | F | CN | OCH₃ | OCH₂CH(CH₃)₂ |
| I.a.145. | H | F | H | OC₂H₄OCH₃ |
| I.a.146. | H | F | CH₃ | OC₂H₄OCH₃ |
| I.a.147. | H | F | OCH₃ | OC₂H₄OCH₃ |
| I.a.148. | H | Cl | H | OC₂H₄OCH₃ |
| I.a.149. | H | Cl | CH₃ | OC₂H₄OCH₃ |
| I.a.150. | H | Cl | OCH₃ | OC₂H₄OCH₃ |
| I.a.151. | H | Br | H | OC₂H₄OCH₃ |
| I.a.152. | H | Br | CH₃ | OC₂H₄OCH₃ |
| I.a.153. | H | Br | OCH₃ | OC₂H₄OCH₃ |
| I.a.154. | H | CN | H | OC₂H₄OCH₃ |
| I.a.155. | H | CN | CH₃ | OC₂H₄OCH₃ |
| I.a.156. | H | CN | OCH₃ | OC₂H₄OCH₃ |
| I.a.157. | F | F | H | OC₂H₄OCH₃ |
| I.a.158. | F | F | CH₃ | OC₂H₄OCH₃ |
| I.a.159. | F | F | OCH₃ | OC₂H₄OCH₃ |
| I.a.160. | F | Cl | H | OC₂H₄OCH₃ |
| I.a.161. | F | Cl | CH₃ | OC₂H₄OCH₃ |
| I.a.162. | F | Cl | OCH₃ | OC₂H₄OCH₃ |
| I.a.163. | F | Br | H | OC₂H₄OCH₃ |
| I.a.164. | F | Br | CH₃ | OC₂H₄OCH₃ |
| I.a.165. | F | Br | OCH₃ | OC₂H₄OCH₃ |
| I.a.166. | F | CN | H | OC₂H₄OCH₃ |
| I.a.167. | F | CN | CH₃ | OC₂H₄OCH₃ |
| I.a.168. | F | CA | OCH₃ | OC₂H₄OCH₃ |

Also preferred are the phenyluracils of formula (I.b), particularly preferred the phenyluracils of formulae (I.b. 1) to (I.b. 168), more preferably (I.b. 1) to (I.b. 144), which differ from the corresponding phenyluracils of formulae (I.a. 1) to (I.a.168), preferably (I.a.1) to (I.a.144), only in that Z is N:

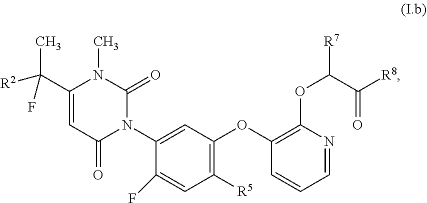

Also preferred are the phenyluracils of formula (I.c), particularly preferred the phenyluracils of formulae (I.c. 1) to (I.c. 168), more preferably (I.c.1) to (I.c. 144), which differ from the corresponding phenyluracils of formulae (I.a.1) to (I.a.168), preferably (I.a.1) to (I.a.144), only in that X is S:

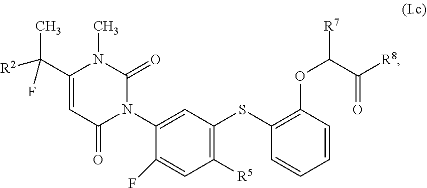

Also preferred are the phenyluracils of formula (I.d), particularly preferred the phenyluracils of formulae (I.d. 1) to (I.d. 168), more preferably (I.d. 1) to (I.d. 144), which differ from the corresponding phenyluracils of formulae (I.a. 1) to (I.a.168), preferably (I.a.1) to (I.a.144), only in that X is S and Z is N:

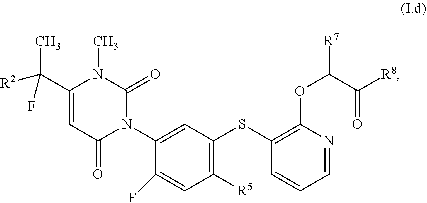

Also preferred are the phenyluracils of formula (I.e), particularly preferred the phenyluracils of formulae (I.e. 1) to (I.e. 168), more preferably (I.e. 1) to (I.e. 144), which differ from the corresponding phenyluracils of formulae (I.a.1) to (I.a.168), preferably (I.a. 1) to (I.a.144), only in that Q is S:

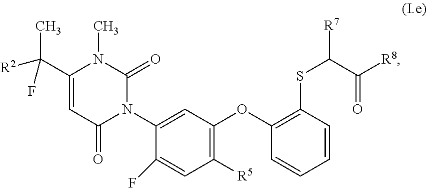

Also preferred are the phenyluracils of formula (I.f), particularly preferred the phenyluracils of formulae (I.f.1) to (I.f. 168), more preferably (I.f.1) to (I.f.144), which differ from the corresponding phenyluracils of formulae (I.a.1) to (I.a.168), preferably (I.a.1) to (I.a.144), only in that Q is S and Z is N:

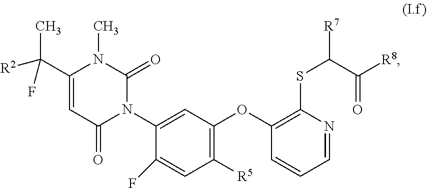

Also preferred are the phenyluracils of formula (I.g), particularly preferred the phenyluracils of formulae (I.g. 1) to (I.g. 168), more preferably (I.g.1) to (I.g. 144), which differ from the corresponding phenyluracils of formulae (I.a.1) to (I.a.168), preferably (I.a.1) to (I.a.144), only in that X and Q are S:

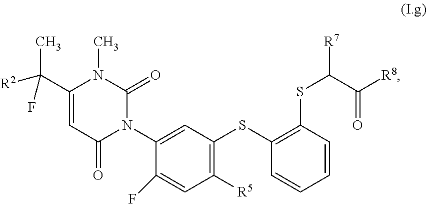

Also preferred are the phenyluracils of formula (I.h), particularly preferred the phenyluracils of formulae (I.h.1) to (I.h. 168), more preferably (I.h.1) to (I.h. 144), which differ from the corresponding phenyluracils of formulae (I.a.1) to (I.a.168), preferably (I.a.1) to (I.a.144), only in that X and Q are S and Z is N:

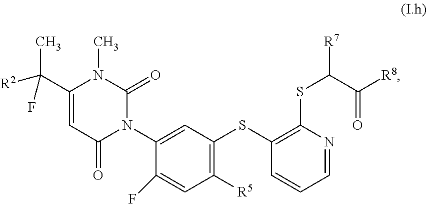

Also preferred are the phenyluracils of formula (I.i) wherein Z is CH or N, i.e. being the phenyluracils of formulae (I.a) and (I.b) as defined above,
particularly preferred the phenyluracils of formulae (I.i. 1) to (I.i. 168) wherein Z is CH or N, i.e. being the phenyluracils of formulae (I.a.1) to (I.a.168) and (I.b. 1) to (I.b. 144) as defined above; more preferably the phenyluracils of formulae (I.i.1) to (I.i. 144) wherein Z is CH or N, i.e. being the phenyluracils of formulae (I.a.1) to (I.a.144) and (I.b.1) to (I.b. 144) as defined above:

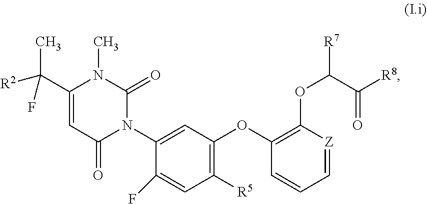

wherein Z is CH or N

The phenyluracils of formula (I) according to the invention can be prepared by standard processes of organic chemistry, for example by the following processes:

### Process A)

The uracilpyridines of formula (I) are obtained from the acid halides of formula (II) by reaction with compounds of formula (III) in the presence of a base:

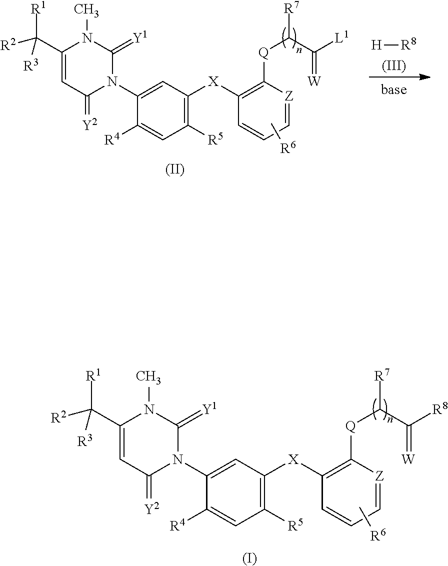

Within the acid halides of formula (II), L¹ is halogen; preferably is F, Cl or Br; especially preferred is F or Cl, more preferred is CI.

Instead of the acid halides of formula (II), also the corresponding acid (e.g. acid halide of formula (II), wherein L¹ is OH) in combination with an activating reagent, like carbonyldiimidazole, N,N′-Dicyclohexylcarbodiimide (DCC), 1-ethyl-3-(3-dimethylaminopropyl)-carbodiimide (EDC) or N-methyl-2-chloropyridinium chloride can be used. The reaction conditions are the same as described for the acid halides of formula (II).

The compounds (III) can also be employed in the form of their salts, in particular the sodium and potassium salts, in which case the presence of a base is not necessary.

The reaction of acid halides (II) with compounds (III) is usually carried out from 0° C. to the boiling point of the reaction mixture, preferably at from 0° C. to 100° C., particularly preferably at from 0° C. to 40° C., in an inert organic solvent in the presence of a base.

The reaction may in principle be carried out in substance. However, preference is given to reacting the acid halides (II) with the compounds (III) in an organic solvent. Suitable in principle are all solvents, which are capable of dissolving the acid halides (II) and the compounds (III) at least partly, and preferably fully under reaction conditions.

Examples of suitable solvents are aliphatic hydrocarbons such as pentane, hexane, cyclohexane, nitromethane and mixtures of C₅-C₈-alkanes; aromatic hydrocarbons such as benzene, chlorobenzene, tolene, cresols, o-, m- and p-xylene; halogenated hydrocarbons such as dichloromethane, 1,2-dichloroethane, chloroform, carbon tetrachloride and chlorobenzene; ethers such as diethyl ether, diisopropyl ether, tert.-butyl methylether (TBME), dioxane, anisole and tetrahydrofuran (THF); esters such as ethyl acetate and butyl acetate; nitriles such as acetonitrile and propionitrile; ketones such as acetone, methyl ethyl ketone, diethyl ketone, tert-butyl methyl ketone, cyclohexanone; dipolar aprotic solvents such as sulfolane, dimethyl-sulfoxide, N,N-dimethylformamide (DMF), N,N-dimethylacetamide (DMAC), 1,3-dimethyl-2-imidazolidinone (DMI), N,N′-dimethyl-propylene urea (DMPU), dimethyl sulfoxide (DMSO) and 1-methyl-2 pyrrolidinone (NMP).

Preferred solvents are halogenated hydrocarbons, ethers and dipolar aprotic solvents as mentioned above.

It is also possible to use mixtures of the solvents mentioned.

Examples of suitable bases include metal-containing bases and nitrogen-containing bases.

Examples of suitable metal-containing bases are inorganic compounds such as alkali metal and alkaline earth metal oxide, and other metal oxides, such as lithium oxide, sodium oxide, potassium oxide, magnesium oxide, calcium oxide and magnesium oxide, iron oxide, silver oxide; alkali metal and alkaline earth metal hydrides such as lithium hydride, sodium hydride, potassium hydride and calcium hydride; alkali metal and alkaline earth metal carbonates such as lithium carbonate, sodium carbonate, potassium carbonate, magnesium carbonate, and calcium carbonate; alkali metal hydrogen carbonates (bicarbonates) such as lithium hydrogen carbonate, sodium hydrogen carbonate, potassium hydrogen carbonate; alkali metal and alkaline earth metal phosphates such as potassium phosphate, calcium phosphate; and furthermore organic bases, such as tertiary amines such as trimethylamine, triethylamine, diisopropylethylamine, tributylamine and N-methylpiperidine, pyridine, substituted pyridines such as collidinge, lutidine, N-methylmorpholine and 4-dimethylaminopyridine and also bicyclic amines.

Examples of suitable nitrogen-containing bases are C1-C6-alkylamines, preferably trialkylamines, for example triethylamine, trimethylamine, N-ethyl-diisopropyl-amine; pyridine, lutidine, collidine, 4-(dimethylamino)pyridine (DMAP), imidazole, 1,8-diazabicyclo[5.4.0]undec-7-ene (DBU) or 1,5-diazabi-cyclo-[4.3.0]-non-5-ene (DBN).

Preferred bases are alkali metal and alkaline earth metal carbonates and nitrogen-containing bases as defined above; especially preferred triethylamine, pyridine or sodium carbonate.

The term base as used herein also includes mixtures of two or more, preferably two of the above compounds. Particular preference is given to the use of one base.

The bases are generally used in excess, more preferably with from 1 to 3 equivalents based on the acid halides (II), and they may also be used as the solvent. However, they can also be employed in catalytic amounts.

For the reaction, the acid halides (II), the compounds (III) and the base can be brought into contact in any way per se.

Accordingly, the reaction partners and the base may be introduced into the reaction vessel and reacted separately, simultaneously or successively.

The reactants are generally employed in equimolar amounts. It might be advantageous using one of the reactants in excess, for example with a view to complete a reaction of the other reactant.

The reaction can be carried out at atmospheric pressure, reduced pressure or under elevated pressure, if appropriate under an inert gas, continuously or batchwise.

The end of the reaction can easily be determined by the skilled worker by means of routine methods.

The reaction mixtures are worked up in a customary manner, for example by mixing with water, separation of the phases and, if appropriate, chromatographic purification of the crude product. Some of the intermediates and end products are obtained in the form of viscous oils, which can be purified or freed from volatile components under reduced pressure and at moderately elevated temperature.

If the intermediates and the end products are obtained as solid, purification can also be carried out by recrystallisation or digestion.

The compounds of formula (III) are commercially available.

### Process B)

As an alternative, the phenyluracils of formula (I), wherein R⁵ is halogen or CN can also be prepared by reacting phenyluracils of formula (I), wherein R⁵ is NH₂, with a diazotizing agent optionally in the presence of copper salts:

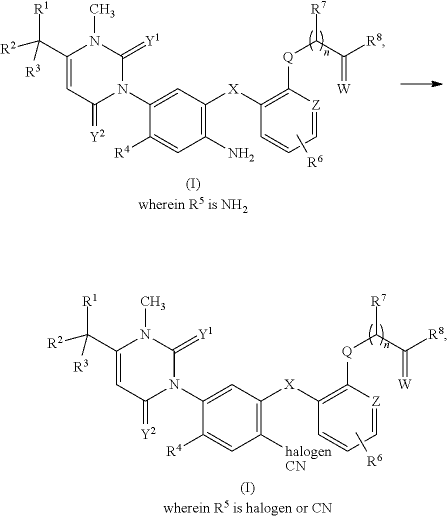

The halogenation of the phenyluracils of formula (I), wherein R⁵ is NH₂, is performed by diazotization with an alkyl nitrite (e.g. isoamyl nitrite, tert-Butyl nitrite or NaNO₂) followed by treatment with a copper (I) and/or copper (II) halide (e.g. CuCl, CuCl₂, CuBr, CuBr₂ or CuCN) in a solvent such as acetonitrile at a temperature ranging from 0° C. to the reflux temperature of the solvent to give the corresponding phenyluracils of formula (I), wherein R⁵ is a halogen, such as chloride or bromide, or CN (e.g. WO 2011/137088).

This reaction is known as “Sandmeyer” reaction (see for example L. Kürti, B. Czako Strategic Applications of Named Reactions in Organic Synthesis, Elsevier: San Diego, 2005, p. 394-395).

To obtain phenyluracils of formula (I), wherein R⁵ is iodine, no copper salts are required.

The below mentioned reaction conditions can be used, adding instead of a copper salt an iodine salt such as potassium iodide after diazotization.

To obtain phenyluracils of formula (I), wherein R⁵ is fluorine, tetrafluoroborate salts of the diazonium compound can be used. These are obtained by adding hydrogene tetrafluoroborate during the diazotization. Subsequent thermal or photolytical decomposition delivers the corresponding fluoro compounds (Langlois, B. In Introduction of Fluorine via Diazonium Compounds (Fluorodediazoniation); Baasner, B., Hagemann, H., Tatlow, J. C., Eds.; Houben-Weyl, Methods of Organic Chemistry; Thieme: Stuttgart, 1999; Vol. E10a, Organo-Fluorine Compounds, pp 686-740).

The reaction of phenyluracils of formula (I), wherein R⁵ is NH₂, with a diazotization agent and optionally copper salts is usually carried out from 0° C. to the boiling point of the reaction mixture, preferably from 0° C. to 100° C., particularly preferably from 0° C. to 40° C., in an inert solvent.

The reaction may in principle be carried out in substance. However, preference is given to reacting the phenyluracil of formula (I), wherein R⁵ is NH₂, with the copper salts and the diazotization agent in an organic solvent.

Suitable in principle are all solvents, which are capable of dissolving the compounds of formula (I), wherein R⁵ is NH₂, the copper salts and the diazotization agent at least partly, and preferably fully under reaction conditions.

Examples of suitable solvents are aliphatic hydrocarbons such as pentane, hexane, cyclohexane, nitromethane and mixtures of C₅-C₈-alkanes; aromatic hydrocarbons such as benzene, chlorobenzene, tolene, cresols, o-, m- and p-xylene; halogenated hydrocarbons such as dichloromethane, 1,2-dichloroethane, chloroform, carbon tetrachloride and chlorobenzene; ethers such as diethyl ether, diisopropyl ether, tert.-butyl methylether (TBME), dioxane, anisole and tetrahydrofuran (THF); esters such as ethyl acetate and butyl acetate; nitriles such as acetonitrile and propionitrile; ketones such as acetone, methyl ethyl ketone, diethyl ketone, tertbutyl methyl ketone, cyclohexanone; dipolar aprotic solvents such as sulfolane, dimethylsulfoxide, N,N-dimethylformamide (DMF), N,N-dimethylacetamide (DMAC), 1,3-dimethyl-2-imidazolidino-ne (DMI), N,N′-dimethylpropylene urea (DMPU), dimethyl sulfoxide (DMSO) and 1-methyl-2 pyrrolidinone (NMP) and polar protic solvents as water.

Preferred solvents are nitriles or polar protic solvents as mentioned above.

It is also possible to use mixtures of the solvents mentioned.

The copper salts are generally used in excess, more preferably with from 1 to 3 equivalents based on the phenyluracil of formula (I), wherein R⁵ is NH₂.

The diazotization agent generally used in excess, more preferably with from 1 to 3 equivalents based on the compounds of formula (I), wherein R⁵ is NH₂.

For the reaction, the phenyluracils of formula (I), wherein R⁵ is NH₂, the copper salts and the diazotization agent can be brought into contact in any way per se.

The reaction can be carried out at atmospheric pressure, reduced pressure or under elevated pressure, if appropriate under an inert gas, continuously or batchwise.

The copper salts and the diazotization agents are commercially available.

Phenyluracils of formula (I) wherein R⁵ is NH₂ can be prepared from phenyluracils of formula (I) wherein R⁵ is NO₂:

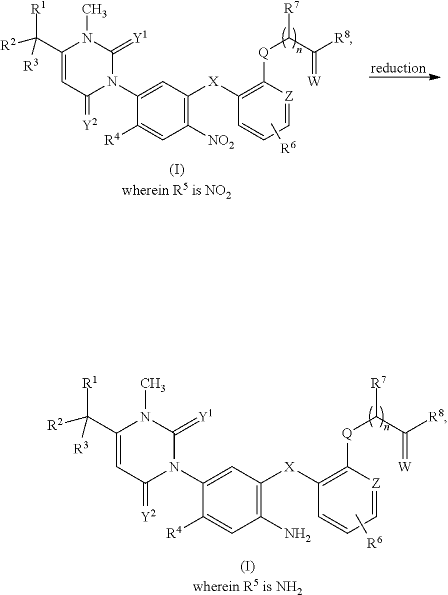

The reduction of the nitro group on can be achieved by treatment with iron powder in acetic acid at a temperature ranging from 0° C. to 100° C. Alternatively, the reduction can be carried out by catalytic hydrogenation in hydrogen gas at a pressure of 70 to 700 kPa, preferably 270 to 350 kPa, in the presence of a metal catalyst such as palladium supported on an inert carrier such as activated carbon, in a weight ratio of 5 to 20% of metal to carrier, suspended in a solvent such as ethanol at ambient temperature (see e.g. WO 2011/137088).

The reaction of phenyluracils of formula (I), wherein R⁵ is NO₂, with the reducing agent is usually carried out from 0° C. to the boiling point of the reaction mixture, preferably from 20° C. to the boiling point of the reaction mixture, in an inert solvent. The reaction may in principle be carried out in substance.

However, preference is given to reacting the compounds of formula (I), wherein R⁵ is NO₂, with the reducing agent in an organic solvent. Suitable in principle are all solvents, which are capable of dissolving the compounds of formula (I), wherein R⁵ is NO₂, at least partly, and preferably fully under reaction conditions.

Examples of suitable solvents are alcohols such as ethanol.

The reducing agents are generally used in excess, more preferably with from 1 to 6 equivalents based on the nitro compounds.

The reaction can be carried out at atmospheric pressure, reduced pressure or under elevated pressure, if appropriate under an inert gas, continuously or batchwise.

The reducing agents are commercially available.

Phenyluracils of formula (I), wherein R⁵ is NO₂ can be prepared by reaction of uracils of formula (IV) with compounds of formula (V) in the presence of a base:

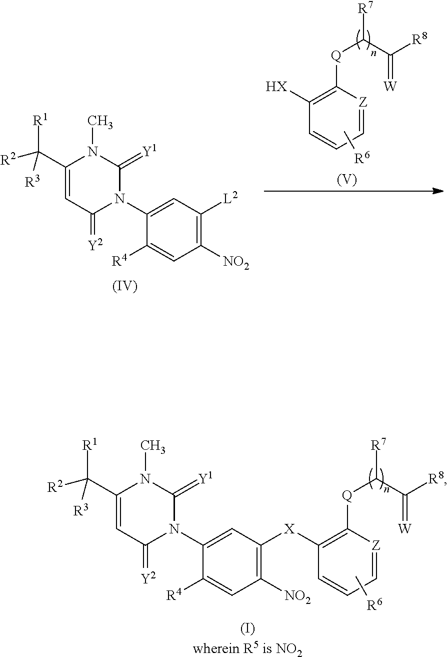

Within the uracils of formula (IV), L² is a leaving group such halogen, C₁-C₆-alkylsulfonate or arylsulfonate; preferably F, C₁-C₆-alkylsulfonate or arylsulfonate; especially preferred F, mesylat or tosylat.

The reaction of the uracils of formula (IV) with compounds of formula (V) in presence of a base is usually carried out from 0° C. to the boiling point of the reaction mixture, preferably from 20° C. to 100° C.

The reaction may in principle be carried out in substance. However, preference is given to reacting the uracils of formula (IV) with the compounds of formula (V) in an organic solvent. Suitable in principle are all solvents which are capable of dissolving the uracils of formula (IV) and the compounds of formula (V) at least partly and preferably fully under reaction conditions.

Examples of suitable solvents are aliphatic hydrocarbons such as pentane, hexane, cyclohexane, nitromethane and mixtures of C₅-C₈-alkanes, aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

Preferred solvents are ethers, nitriles and dipolar aprotic solvents as mentioned above. Examples of suitable metal-containing bases are inorganic compounds such as alkali metal and alkaline earth metal hydroxides, and other metal hydroxides, such as lithium hydroxide, sodium hydroxide, potassium hydroxide, magnesium hydroxide, calcium hydroxide and aluminum hydroxide; alkali metal and alkaline earth metal oxide, and other metal oxides, such as lithium oxide, sodium oxide, potassium oxide, magnesium oxide, calcium oxide and magnesium oxide, iron oxide, silver oxide; alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, sodium carbonate, potassium carbonate, magnesium carbonate, cesium carbonate and calcium carbonate, as well as alkali metal hydrogen carbonates (bicarbonates) such as lithium hydrogen carbonate, sodium hydrogen carbonate, potassium hydrogen carbonate; alkali metal and alkaline earth metal phosphates such as potassium phosphate, calcium phosphate; metal organic compounds, preferably alkali metal alkyls such as methyl lithium, butyl lithium and phenyl lithium, alkyl magnesium halides such as methyl magnesium chloride as well as alkali metal and alkaline earth metal alkoxides such as sodium methoxide, sodium ethoxide, potassium ethoxide, potassium tert-butoxide, potassium tert-pentoxide and di-methoxymagnesium.

Compounds of formula (V) are known from literature and/or commercially available.

Uracils of formula (IV) can be prepared by reaction of uracils of formula (VI) with compounds of formula (VII) in the presence of a base:

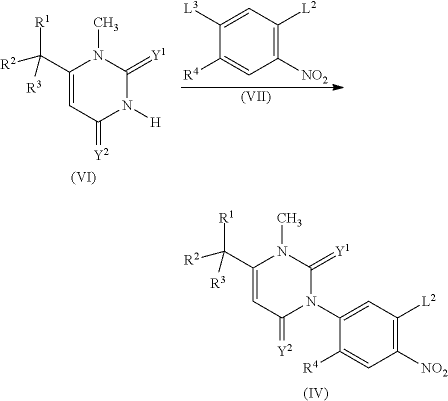

Within the compounds of formula (VII), L² is a leaving group such halogen, C₁-C₆-alkylsulfonate or arylsulfonate; preferably F, C₁-C₆-alkylsulfonate or arylsulfonate; especially preferred F, mesylat or tosylat.

Within the compounds of formula (VII), L³ is a leaving group such F, C₁-C₆-alkylsulfonate or arylsulfonate; preferably F, mesylat or tosylat.

The reaction of the uracils of formula (VI) with compounds of formula (VII) in presence of a base is usually carried out from 0° C. to the boiling point of the reaction mixture, preferably from 20° C. to 100° C.

The reaction may in principle be carried out in substance. However, preference is given to reacting the uracils of formula (VI) with the compounds of formula (VII) in an organic solvent. Suitable in principle are all solvents which are capable of dissolving the uracils of formula (VI) and the compounds of formula (VII) at least partly and preferably fully under reaction conditions. Examples of suitable solvents are dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

Examples of suitable metal-containing bases are inorganic compounds such as alkali metal and alkaline earth metal hydroxides, and other metal hydroxides, such as lithium hydroxide, sodium hydroxide, potassium hydroxide, magnesium hydroxide, calcium hydroxide and aluminum hydroxide; alkali metal and alkaline earth metal oxide, and other metal oxides, such as lithium oxide, sodium oxide, potassium oxide, magnesium oxide, calcium oxide and magnesium oxide, iron oxide, silver oxide; alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, sodium carbonate, potassium carbonate, magnesium carbonate, cesium carbonate and calcium carbonate, as well as alkali metal hydrogen carbonates (bicarbonates) such as lithium hydrogen carbonate, sodium hydrogen carbonate, potassium hydrogen carbonate; alkali metal and alkaline earth metal phosphates such as potassium phosphate, calcium phosphate; metal organic compounds, preferably alkali metal alkyls such as methyl lithium, butyl lithium and phenyl lithium, alkyl magnesium halides such as methyl magnesium chloride as well as alkali metal and alkaline earth metal alkoxides such as sodium methoxide, sodium ethoxide, potassium ethoxide, potassium tert-butoxide, potassium tert-pentoxide and dimethoxymagnesium.

Compounds of formula (VII) are known from literature and/or commercially available.

Uracils of formula (VI) can be prepared from NH-uracils of formula (VIII) using methylation reagents, such as methyl iodide, methyl sulfates or methyl sulfonates, e.g. CF₃SO₃CH₃, in the presence of a base:

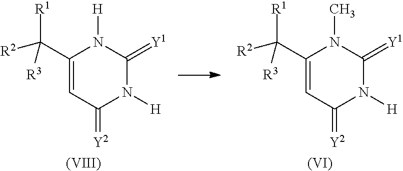

The reaction may in principle be carried out in substance. However, preference is given to reacting the uracils of formula (VIII) in an organic solvent.

Suitable in principle are all solvents which are capable of dissolving the uracils of formula (VIII) at least partly and preferably fully under reaction conditions.

Examples of suitable solvents are ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

Examples of suitable metal-containing bases are alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, sodium carbonate, potassium carbonate, magnesium carbonate, cesium carbonate and calcium carbonate, as well as alkali metal hydrogen carbonates (bicarbonates) such as lithium hydrogen carbonate, sodium hydrogen carbonate, potassium hydrogen carbonate; alkali metal and alkaline earth metal phosphates such as potassium phosphate, calcium phosphate; metal organic compounds, preferably alkali metal alkyls such as methyl lithium, butyl lithium and phenyl lithium, alkyl magnesium halides such as methyl magnesium chloride as well as alkali metal and alkaline earth metal alkoxides such as potassium tert-butoxide.

NH-Uracils of formula (VIII) can be prepared from aminocrotonates of formula (IX) using isocyanate salts, such as KNCO:

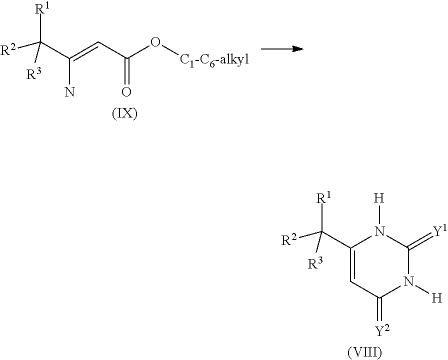

The reaction may in principle be carried out in substance. However, preference is given to reactions in an organic solvent.

Suitable in principle are all solvents which are capable of dissolving the aminocrotonate of formula (IX) and the isocyanate salt at least partly and preferably fully under reaction conditions. Examples of suitable solvents are ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

Aminocrotonates of formula (IX) can be prepared from beta-keto-esters of formula (X) as described in the literature (see for example US 2003/0216594):

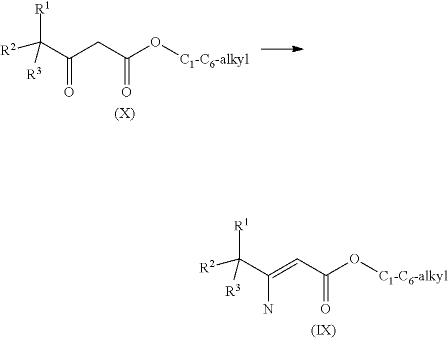

Beta-keto-esters of formula (X) can be prepared by reaction of esters of formula (XI) with an acetic acid ester of formula (XII) in the presence of a base:

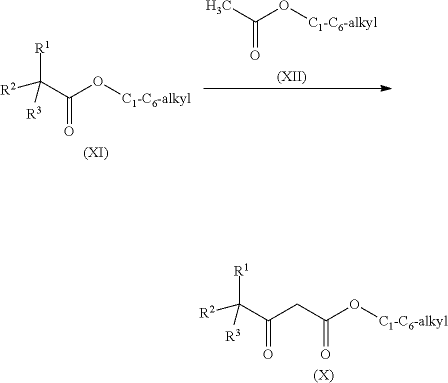

The reaction may in principle be carried out in substance. However, preference is given to a reaction in an organic solvent.

Suitable in principle are all solvents which are capable of dissolving esters of formula (XI) at least partly and preferably fully under reaction conditions.

Examples of suitable solvents are ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

Examples of suitable metal-containing bases are alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, sodium carbonate, potassium carbonate, magnesium carbonate, cesium carbonate and calcium carbonate, as well as alkali metal hydrogen carbonates (bicarbonates) such as lithium hydrogen carbonate, sodium hydrogen carbonate, potassium hydrogen carbonate; alkali metal and alkaline earth metal phosphates such as potassium phosphate, calcium phosphate; metal organic compounds, preferably alkali metal alkyls such as methyl lithium, butyl lithium and phenyl lithium, alkyl magnesium halides such as methyl magnesium chloride as well as alkali metal and alkaline earth metal alkoxides such as potassium tert-butoxide.

Compounds of formula (XI) and (XII) are known from literature and/or commercially available.

### Process C)

The phenyluracils of formula (I) can also be prepared by reaction of compounds of formula (XIII) with alkylating agents of formula (XIV) in the presence of a base in analogy to known processes (e.g. WO 11/137088):

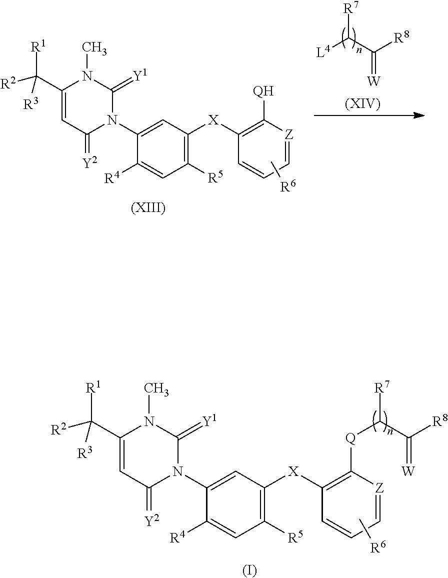

Within the alkylating agents of formula (XIV), L⁴ is a leaving group such halogen, C₁-C₆-alkylsulfonate or arylsulfonate; preferably CI or Br.

The reaction may in principle be carried out in substance. However, preference is given to reacting the compounds of formula (XIII) with the alkylating agents of formula (XIV) in an organic solvent.

Suitable in principle are all solvents which are capable of dissolving the compounds of formula (XIII) and the alkylating agents of formula (XIV) at least partly and preferably fully under reaction conditions.

Examples of suitable solvents are ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, as well as dipolar aprotic solvents such as sulfolane, dimethylsulfoxide, N,N-dimethylformamide (DMF), N,N-dimethyl-acetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

Examples of suitable metal-containing bases are inorganic compounds such as alkali metal and alkaline earth metal hydroxides, and other metal hydroxides, such as lithium hydroxide, sodium hydroxide, potassium hydroxide, magnesium hydroxide, calcium hydroxide and aluminum hydroxide; alkali metal and alkaline earth metal oxide, and other metal oxides, such as lithium oxide, sodium oxide, potassium oxide, magnesium oxide, calcium oxide and magnesium oxide, iron oxide, silver oxide; alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, sodium carbonate, potassium carbonate, magnesium carbonate, cesium carbonate and calcium carbonate, as well as alkali metal hydrogen carbonates (bicarbonates) such as lithium hydrogen carbonate, sodium hydrogen carbonate, potassium hydrogen carbonate; alkali metal and alkaline earth metal phosphates such as potassium phosphate, calcium phosphate; metal organic compounds, preferably alkali metal alkyls such as methyl lithium, butyl lithium and phenyl lithium, alkyl magnesium halides such as methyl magnesium chloride as well as alkali metal and alkaline earth metal alkoxides such as sodium methoxide, sodium ethoxide, potassium ethoxide, potassium tert-butoxide, potassium tert-pentoxide and dimethoxymagnesium; and furthermore organic bases, such as tertiary amines such as trimethylamine, triethylamine, diisopropylethylamine and N-methylpiperidine, pyridine, substituted pyridines such as collidine, lutidine, N-methylmorpholine and 4-dimethylaminopyridine and also bicyclic amines.

The alkylating agents of formula (XIV) are commercially available or can be prepared by known methods (e.g. Lowell, Andrew N. et al, Tetrahedron, 6(30), 5573-5582, 2010; WO 11/137088).

Compounds of formula (XIII) can be prepared as follows:

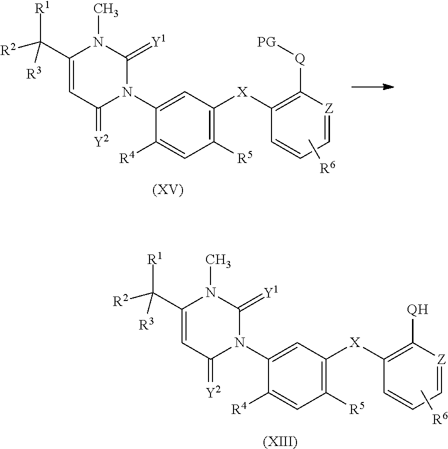

Within the compounds of formula (XV) “PG” is a protecting group selected from the group consisting of C₁-C₆-alkyl, C₁-C₆-cyanoalkyl, C₁-C₆-haloalkyl, C₁-C₆-alkylthio-C₁-C₄-alkyl, C₁-C₆-alkoxy-C₁-C₄-alkyl, C₁-C₆-alkoxy-C₁-C₄-alkoxy-C₁-C₄-alkyl, (tri-C₁-C₆-alkyl)silyl-C₁-C₄-alkyl, (tri-C₁-C₆-alkyl)silyl-C₁-C₄-alkyoxy-C₁-C₄-alkyl, C₂-C₆-alkenyl, C₃-C₆-alkynyl, C₃-C₆-cycloalkyl, C₃-C₆-cylcloalkyl-C₁-C₄-alkyl, C₅-C₆-cycloalkenyl, tetrahydropyranyl, (tri-C₁-C₆-alkyl)silyl, [(diphenyl)(C₁-C₄-alkyl)]silyl, formyl, C₁-C₆-alkyl-carbonyl, C₁-C₆-alkyl-O-carbonyl, C₂-C₆-alkenyl-Ocarbonyl, [(diphenyl)(C₁-C₄-alkyl)]silyl-C₁-C₄-alkyl, phenyl-C₁-C₄-alkyl, phenylthio-C₁-C₆-alkyl, phenylcarbonyl, wherein each phenyl ring can be substituted by one to three substituents selected from the group consisting of halogen, CN, NO2, C1-C4-alkyl and C1-C4-alkoxy. Preferably PG is C₁-C₆-alkyl, C₁-C₆-alkoxy-C₁-C₄-alkyl, (tri-C₁-C₆-alkyl)silyl-C1-C4-alkyl, C₂-C₆-alkenyl, tetrahydropyranyl, (tri-C₁-C₆-alkyl)silyl, [(diphenyl)(C₁-C₄-alkyl)]silyl or phenyl-C₁-C₄-alkyl.

For example, the compounds of formula (XIII) can be prepared by treating the compounds of formula (XV), wherein “PG” is methyl, with boron tribromide in a solvent such as dichloromethane, acetonitrile or 1,4-dioxane, or without a solvent at temperatures ranging from 0° C. to 150° C.

Alternatively, compounds of formula (XIII) can be prepared by deprotecting compounds of formula (XV), wherein “PG” is a benzyl group, by catalytic hydrogenation in a hydrogen gas atmosphere at a pressure of 70 to 700 kPa, preferably 270 to 350 kPa, in the presence of a metal catalyst such as palladium supported on an inert carrier such as activated carbon, in a weight ratio of 5 to 20% of metal to carrier, suspended in a solvent such as ethanol at ambient temperature. The process, use and choice of the protecting groups will be apparent to one skilled in chemical synthesis (see, for example, Greene, T. W.; Wuts, P. G. M. Protective Groups in Organic Synthesis, 4th ed.; Wiley: New York, 2007).

Compounds of formula (XV) can be prepared from amino compounds of formula (XVI) using the “Sandmeyer” reaction as described in Process B:

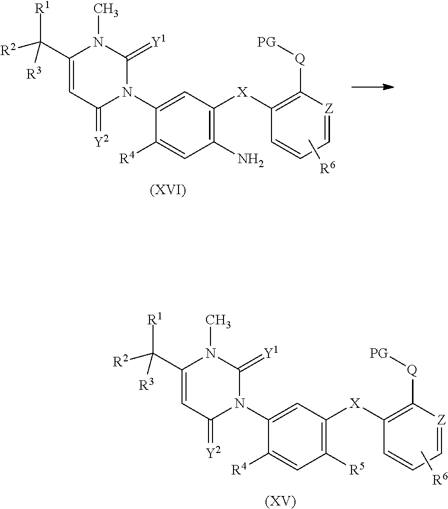

Amino compounds of formula (XVI) can be prepared from nitro compounds of formula (XVII) using reduction conditions as described in Process B:

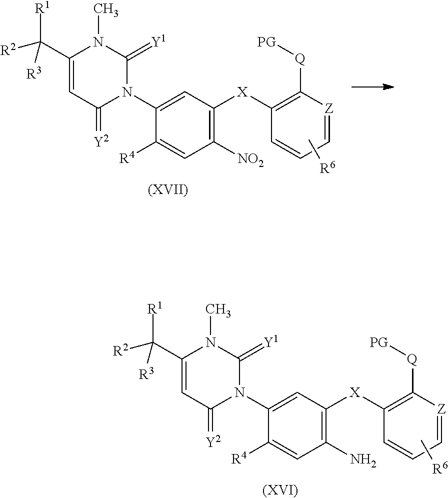

Nitro compounds of formula (XVII) can be prepared from uracils of formula (IV) in the presence of a base using compounds of formula (XVIII), as described in process B:

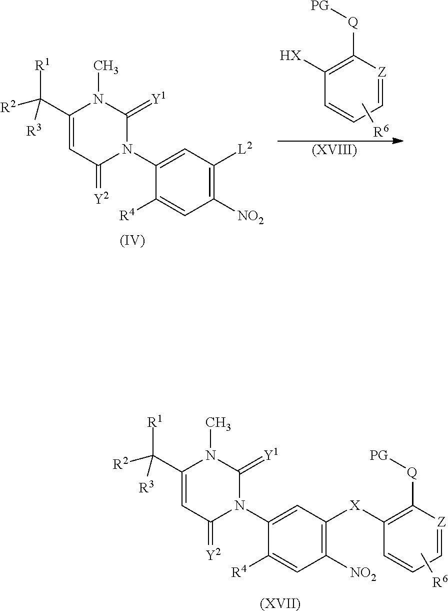

Compounds of formula (XVIII) are known to literature and/or commercially available.

To widen the spectrum of action and to achieve synergistic effects, the phenyluracils of formula (I) may be mixed with many representatives of other herbicidal or growth-regulating active ingredient groups and then applied concomitantly. Suitable components for combinations are, for example, herbicides from the classes of the acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas.

It may furthermore be beneficial to apply the phenyluracils of formula (I) alone or in combination with other herbicides, or else in the form of a mixture with other crop protection agents, for example together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

The invention also relates to formulations comprising at least an auxiliary and at least one phenyluracil of formula (I) according to the invention.

A formulation comprises a pesticidal effective amount of a phenyluracil of formula (I). The term “effective amount” denotes an amount of the combination or of the phenyluracils of formula (I), which is sufficient for controlling undesired vegetation, especially for controlling undesired vegetation in crops (i.e. cultivated plants) and which does not result in a substantial damage to the treated crop plants. Such an amount can vary in a broad range and is dependent on various factors, such as the undesired vegetation to be controlled, the treated crop plants or material, the climatic conditions and the specific phenyluracil of formula (I) used.

The phenyluracils of formula (I), their salts amides, esters or thioesters can be converted into customary types of formulations, e.g. solutions, emulsions, suspensions, dusts, powders, pastes, granules, pressings, capsules, and mixtures thereof. Examples for formulation types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further formulation types are defined in the “Catalogue of pesticide formulation types and international coding system”, Technical Monograph No. 2, 6^{th} Ed. May 2008, CropLife International.

The formulations are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetting agents, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in Mccutcheon's, Vol. 1: Emulsifiers & Detergents, Mccutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the phenyluracils of formula (I) on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin. Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for formulation types and their preparation are:

### i) Water-Soluble Concentrates (SL, LS)

10-60 wt % of a phenyluracil of formula (I) according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

### ii) Dispersible Concentrates (DC)

5-25 wt % of a phenyluracil of formula (I) according to the invention and 1-10 wt % dispersant (e.g. polyvinylpyrrolidone) are dissolved in organic solvent (e.g. cyclohexanone) ad 100 wt %. Dilution with water gives a dispersion.

iii) Emulsifiable Concentrates (EC)

15-70 wt % of aphenyluracil of formula (I) according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

### iv) Emulsions (EW, EO, ES)

5-40 wt % of a phenyluracil of formula (I) according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a phenyluracil of formula (I) according to the invention are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type formulation up to 40 wt % binder (e.g. polyvinylalcohol) is added.

### vi) Water-Dispersible Granules and Water-Soluble Granules (WG, SG)

50-80 wt % of a phenyluracil of formula (I) according to the invention are ground finely with addition of dispersants and wetting agents (e.g. sodium lignosulfonate and alcohol ethoxylate) ad 100 wt % and prepared as water-dispersible or water-soluble granules by means of technical appliances (e.g. extrusion, spray tower, fluidized bed). Dilution with water gives a stable dispersion or solution of the active substance.

vii) Water-Dispersible Powders and Water-Soluble Powders (WP, SP, WS)

50-80 wt % of a phenyluracil of formula (I) according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a phenyluracil of formula (I) according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

### iv) Microemulsion (ME)

5-20 wt % of a phenyluracil of formula (I) according to the invention are added to 5-30 wt % organic solvent blend (e.g. fatty acid dimethylamide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion.

### iv) Microcapsules (CS)

An oil phase comprising 5-50 wt % of a phenyluracil of formula (I) according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a phenyluracil of formula (I) according to the invention, 0-40 wt % water insoluble organic solvent (e.g. aromatic hydrocarbon), and an isocyanate monomer (e.g. diphenylmethene-4,4′-diisocyanate) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS formulation.

### ix) Dustable Powders (DP, DS)

1-10 wt % of a phenyluracil of formula (I) according to the invention are ground finely and mixed intimately with solid carrier (e.g. finely divided kaolin) ad 100 wt %.

### x) Granules (GR, FG)

0.5-30 wt % of a phenyluracil of formula (I) according to the invention is ground finely and associated with solid carrier (e.g. silicate) ad 100 wt %. Granulation is achieved by extrusion, spray-drying or the fluidized bed.

### xi) Ultra-Low Volume Liquids (UL)

1-50 wt % of a phenyluracil of formula (I) according to the invention are dissolved in organic solvent (e.g. aromatic hydrocarbon) ad 100 wt %.

The formulation types i) to xi) may optionally comprise further auxiliaries, such as 0.1-1 wt % bactericides, 5-15 wt % anti-freezing agents, 0, 1-1 wt % anti-foaming agents, and 0.1-1 wt % colorants.

The formulations generally comprise between 0.01 and 95%, preferably between 0.1 and 90%, and in particular between 0.5 and 75%, by weight of the phenyluracil of formula (I).

The phenyluracils of formula (I) are employed in a purity of from 90% to 100%, preferably from 95% to 100% (according to NMR spectrum).

Solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The formulations in question give, after two-to-tenfold dilution, active substance concentrations of from 0.01 to 60% by weight, preferably from 0.1 to 40% by weight, in the ready-to-use preparations.

Methods for applying phenyluracils of formula (I), formulations thereof, on to plant propagation material, especially seeds, include dressing, coating, pelleting, dusting, soaking and in-furrow application methods of the propagation material. Preferably, phenyluracils of formula (I), formulations thereof, respectively, are applied on to the plant propagation material by a method such that germination is not induced, e.g. by seed dressing, pelleting, coating and dusting.

Various types of oils, wetting agents, adjuvants, fertilizer, or micronutrients, and further pesticides (e.g. herbicides, insecticides, fungicides, growth regulators, safeners) may be added to the phenyluracils of formula (I), the formulations comprising them as premix or, if appropriate not until immediately prior to use (tank mix). These agents can be admixed with the formulations according to the invention in a weight ratio of 1:100 to 100:1, preferably 1:10 to 10:1.

The user applies the phenyluracils of formula (I) according to the invention, the formulations comprising them usually from a pre-dosage device, a knapsack sprayer, a spray tank, a spray plane, or an irrigation system. Usually, the formulation is made up with water, buffer, and/or further auxiliaries to the desired application concentration and the ready-to-use spray liquor or the formulation according to the invention is thus obtained. Usually, 20 to 2000 liters, preferably 50 to 400 liters, of the ready-to-use spray liquor are applied per hectare of agricultural useful area.

According to one embodiment, either individual components of the formulation according to the invention or partially premixed components, e.g. components comprising phenyluracils of formula (I) may be mixed by the user in a spray tank and further auxiliaries and additives may be added, if appropriate.

In a further embodiment, individual components of the formulation according to the invention such as parts of a kit or parts of a binary or ternary mixture may be mixed by the user himself in a spray tank and further auxiliaries may be added, if appropriate.

In a further embodiment, either individual components of the formulation according to the invention or partially premixed components, e.g. components comprising phenyluracils of formula (I), can be applied jointly (e.g. after tank mix) or consecutively.

The phenyluracils of formula (I), are suitable as herbicides. They are suitable as such or as an appropriately formulation.

The phenyluracils of formula (I) control undesired vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

The phenyluracils of formula (I) have an outstanding herbicidal activity against undesired vegetation, i.e. against a broad spectrum of economically important harmful monocotyledonous and dicotyledonous weeds.

Mentioned below are some representatives of monocotyledonous and dicotyledonous weeds, which can be controlled by the phenyluracils of formula (I) without the enumeration being a restriction to certain species.

Preferably the phenyluracils of formula (I) are used to control monocotyledonous weeds.

Examples of monocotyledonous weeds on which the phenyluracils of formula (I) act efficiently are selected from the genera Hordeum spp., Echinochloa spp., Poa spp., Bromus spp., Digitaria spp., Eriochloa spp., Setaria spp., Pennisetum spp., Eleusine spp., Eragrostis spp., Panicum spp., Lolium spp., Brachiaria spp., Leptochloa spp., Avena spp., Cyperus spp., Axonopris spp., Sorghum spp., and Melinus spp.

Preferred examples of monocotyledonous weeds on which the phenyluracils of formula (I) act efficiently are selected from the species Hordeum murinum, Echinochloa crus-galli, Poa annua, Bromus rubens L., Bromus rigidus, Bromus secalinus L., Digitaria sanguinalis, Digitaria insularis, Eriochloa gracilis, Setaria faberi, Setaria viridis, Pennisetum glaucum, Eleusine indica, Eragrostis pectinacea, Panicum miliaceum, Lolium multiflorum, Brachiaria platyphylla, Leptochloa fusca, Avena fatua, Cyperus compressus, Cyperus esculentes, Axonopris offinis, Sorghum halapense, and Melinus repens.

Especially preferred examples of monocotyledonous weeds on which the phenyluracils of formula (I) act efficiently are selected from the species Echinochloa spp., Digitaria spp., Setaria spp., Eleusine spp. and Brachiarium spp.

Also preferably the phenyluracils of formula (I) are used to control dicotyledonous weeds.

Examples of dicotyledonous weeds on which the phenyluracils of formula (I) act efficiently are selected from the genera Amaranthus spp., Erigeron spp., Conyza spp., Polygonum spp., Medicago spp., Mollugo spp., Cyclospermum spp., Stellaria spp., Gnaphalium spp., Taraxacum spp., Oenothera spp., Amsinckia spp., Erodium spp., Erigeron spp., Senecio spp., Lamium spp., Kochia spp., Chenopodium spp., Lactuca spp., Malva spp., Ipomoea spp., Brassica spp., Sinapis spp., Urtica spp., Sida spp, Portulaca spp., Richardia spp., Ambrosia spp., Calandrinia spp., Sisymbrium spp., Sesbania spp., Capsella spp., Sonchus spp., Euphorbia spp., Helianthus spp., Coronopus spp., Salsola spp., Abutilon spp., Vicia spp., Epilobium spp., Cardamine spp., Picris spp., Trifolium spp., Galinsoga spp., Epimedium spp., Marchantia spp., Solanum spp., Oxalis spp., Metricaria spp., Plantago spp., Tribulus spp., Cenchrus spp. Bidens spp., Veronica spp., and Hypochaeris spp.

Preferred examples of dicotyledonous weeds on which the phenyluracils of formula (I) act efficiently are selected from the species Amaranthus spinosus, Polygonum convolvulus, Medicago polymorpha, Mollugo verticillata, Cyclospermum leptophyllum, Stellaria media, Gnaphalium purpureum, Taraxacum officinale, Oenothera laciniata, Amsinckia intermedia, Erodium cicutarium, Erodium moschatum, Erigeron bonariensis (Conyza bonariensis), Senecio vulgaris, Lamium amplexicaule, Erigeron canadensis, Polygonum aviculare, Kochia scoparia, Chenopodium album, Lactuca serriola, Malva parviflora, Malva neglecta, Ipomoea hederacea, Ipomoea lacunose, Brassica nigra, Sinapis arvensis, Urtica dioica, Amaranthus blitoides, Amaranthus retroflexus, Amaranthus hybridus, Amaranthus lividus, Sida spinosa, Portulaca oleracea, Richardia scabra, Ambrosia artemisiifolia, Calandrinia caulescens, Sisymbrium irio, Sesbania exaltata, Capsella bursa-pastoris, Sonchus oleraceus, Euphorbia maculate, Helianthus annuus, Coronopus didymus, Salsola tragus, Abutilon theophrasti, Vicia benghalensis L., Epilobium paniculatum, Cardamine spp, Picris echioides, Trifolium spp., Galinsoga spp., Epimedium spp., Marchantia spp., Solanum spp., Oxalis spp., Metricaria matriccarioides, Plantago spp., Tribulus terrestris, Salsola kali, Cenchrus spp., Bidens bipinnata, Veronica spp., and Hypochaeris radicata.

Especially preferred examples of dicotyledonous weeds on which the phenyluracils of formula (I) are selected from the species Amaranthus spp., Erigeron spp., Conyza spp., Kochia spp. and Abutilon spp.

The phenyluracils of formula (I), or the formulations comprising them, are applied to the plants mainly by spraying the leaves. Here, the application can be carried out using, for example, water as carrier by customary spraying techniques using spray liquor amounts of from about 100 to 1000 l/ha (for example from 300 to 400 l/ha). The phenyluracils of formula (I), or the formulations comprising them, may also be applied by the low-volume or the ultra-low-volume method, or in the form of microgranules.

Application of the phenyluracils of formula (I), or the formulations comprising them, can be done before, during and/or after, preferably during and/or after, the emergence of the undesired vegetation.

Application of the phenyluracils of formula (I), or the formulations can be carried out before or during sowing.

The phenyluracils of formula (I), or the formulations comprising them, can be applied pre-, post-emergence or pre-plant, or together with the seed of a crop plant. It is also possible to apply the phenyluracils of formula (I), or the formulations comprising them, by applying seed, pretreated with the phenyluracils of formula (I), or the formulations comprising them, of a crop plant. If the active ingredients are less well tolerated by certain crop plants, application techniques may be used in which the combinations are sprayed, with the aid of the spraying equipment, in such a way that as far as possible they do not come into contact with the leaves of the sensitive crop plants, while the active ingredients reach the leaves of undesired vegetation growing underneath, or the bare soil surface (post-directed, lay-by).

In a further embodiment, the phenyluracils of formula (I), or the formulations comprising them, can be applied by treating seed. The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the phenyluracils of formula (I), or the formulations prepared therefrom. Here, the combinations can be applied diluted or undiluted.

The term “seed” comprises seed of all types, such as, for example, corns, seeds, fruits, tubers, seedlings and similar forms. Here, preferably, the term seed describes corns and seeds. The seed used can be seed of the crop plants mentioned above, but also the seed of transgenic plants or plants obtained by customary breeding methods.

When employed in plant protection, the amounts of active substances applied, i.e. the phenyluracils of formula (I) without formulation auxiliaries, are, depending on the kind of effect desired, from 0.001 to 2 kg per ha, preferably from 0.002 to 1 kg per ha, more preferably from 0.005 to 0.5 kg per ha and in particular from 0.01 to 0.2 kg per ha.

In another embodiment of the invention, the application rate of the phenyluracils of formula (I) is from 0.001 to 3 kg/ha, preferably from 0.005 to 2.5 kg/ha and in particular from 0.01 to 2 kg/ha of active substance (a.s.).

In another preferred embodiment of the invention, the rates of application of the phenyluracils of formula (I) according to the present invention (total amount of phenyluracils of formula (I)) are from 0.1 g/ha to 3000 g/ha, preferably 10 g/ha to 1000 g/ha, depending on the control target, the season, the target plants and the growth stage.

In another preferred embodiment of the invention, the application rates of the phenyluracils of formula (I) are in the range from 0.1 g/ha to 5000 g/ha and preferably in the range from 1 g/ha to 2500 g/ha or from 5 g/ha to 2000 g/ha.

In another preferred embodiment of the invention, the application rate of the phenyluracils of formula (I) is 0.1 to 1000 g/ha, preferably 1 to 750 g/ha, more preferably 5 to 500 g/ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

In another embodiment of the invention, to treat the seed, the amounts of active substances applied, i.e. the phenyluracils of formula (I) are generally employed in amounts of from 0.001 to 10 kg per 100 kg of seed.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Depending on the application method in question, the phenyluracils of formula (I), or the formulations comprising them, can additionally be employed in a further number of crop plants for eliminating undesired vegetation.

According to the invention all the crop plants (cultivated plants) mentioned herein are understood to comprise all species, subspecies, variants and/or hybrids which belong to the respective cultivated plants, including but not limited to winter and spring varieties, in particular in cereals such as wheat and barley, as well as oilseed rape, e.g. winter wheat, spring wheat, winter barley etc.

For example, corn is also known as Indian corn or maize (Zea mays) which comprises all kinds of corn such as field corn and sweet corn. According to the invention all maize or corn subspecies and/or varieties are comprised, in particular flour corn (Zea mays var. amylacea), popcorn (Zea mays var. everta), dent corn (Zea mays var. indentata), flint corn (Zea mays var. indurata), sweet corn (Zea mays var. saccharata and var. rugosa), waxy corn (Zea mays var, ceratina), amylomaize (high amylose Zea mays varieties), pod corn or wild maize (Zea mays var. tunicata) and striped maize (Zea mays var. japonica).

Further, most soybean cultivars are classifiable into indeterminate and determinate growth habit, whereas Glycine soja, the wild progenitor of soybean, is indeterminate (PNAS 2010, 107 (19) 8563-856). The indeterminate growth habit (Maturity Group, MG 00 to MG 4.9) is characterized by a continuation of vegetative growth after flowering begins whereas determinate soybean varieties (Maturity Group, (MG) 5 to MG 8) characteristically have finished most of their vegetative growth when flowering begins. According to the invention all soybean cultivars or varieties are comprised, in particular indeterminate and determinate cultivars or varieties.

Examples of suitable crops are the following:
Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera and Zea mays.

Preferred crops are Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N. rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (s. vulgare), Triticale, Triticum aestivum, Triticum durum, Vicia faba, Vitis vinifera and Zea mays.

Especially preferred crops are crops of cereals, corn, soybeans, rice, oilseed rape, cotton, potatoes, peanuts or permanent crops.

The phenyluracils of formula (I) according to the invention, or the formulations comprising them, can also be used in crops which have been modified by mutagenesis or genetic engineering in order to provide a new trait to a plant or to modify an already present trait.

The term “crops” as used herein includes also (crop) plants which have been modified by mutagenesis or genetic engineering in order to provide a new trait to a plant or to modify an already present trait.

Mutagenesis includes techniques of random mutagenesis using X-rays or mutagenic chemicals, but also techniques of targeted mutagenesis, in order to create mutations at a specific locus of a plant genome. Targeted mutagenesis techniques frequently use oligonucleotides or proteins like CRISPR/Cas, zinc-finger nucleases, TALENs or meganucleases to achieve the targeting effect.

Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant in order to add a trait or improve a trait. These integrated genes are also referred to as transgenes in the art, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include in particular herbicide tolerance, insect resistance, increased yield and tolerance to abiotic conditions, like drought.

Herbicide tolerance has been created by using mutagenesis as well as using genetic engineering. Plants which have been rendered tolerant to acetolactate synthase (ALS) inhibitor herbicides by conventional methods of mutagenesis and breeding comprise plant varieties commercially available under the name Clearfield®. However, most of the herbicide tolerance traits have been created via the use of transgenes.

Herbicide tolerance has been created to glyphosate, glufosinate, 2,4-D, dicamba, oxynil herbicides, like bromoxynil and ioxynil, sulfonylurea herbicides, ALS inhibitor herbicides and 4-hydroxyphenylpyruvate dioxygenase (HPPD) inhibitors, like isoxaflutole and mesotrione.

Transgenes which have been used to provide herbicide tolerance traits comprise: for tolerance to glyphosate: cp4 epsps, epsps grg23ace5, mepsps, 2mepsps, gat4601, gat4621 and goxv247, for tolerance to glufosinate: pat and bar, for tolerance to 2,4-D: aad-1 and aad-12, for tolerance to dicamba: dmo, for tolerance to oxynil herbicies: bxn, for tolerance to sulfonylurea herbicides: zm-hra, csr1-2, gm-hra, S4-HrA, for tolerance to ALS inhibitor herbicides: csr1-2, for tolerance to HPPD inhibitor herbicides: hppdPF, W336 and avhppd-03.

Transgenic corn events comprising herbicide tolerance genes are for example, but not excluding others, DAS40278, MON801, MON802, MON809, MON810, MON832, MON87411, MON87419, MON87427, MON88017, MON89034, NK603, GA21, MZHG0JG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC1507 and TC6275.

Transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS81419-2, GU262, SYHTØH2, W62, W98, FG72 and CV127.

Transgenic cotton events comprising herbicide tolerance genes are for example, but not excluding others, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40.

Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants. Transgenes which have most frequently been used are toxin genes of Bacillus spec. and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. However, also genes of plant origin have been transferred to other plants. In particular genes coding for protease inhibitors, like CpTI and pinII. A further approach uses transgenes in order to produce double stranded RNA in plants to target and downregulate insect genes. An example for such a transgene is dvsnf7.

Transgenic corn events comprising genes for insecticidal proteins or double stranded RNA are for example, but not excluding others, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098.

Transgenic soybean events comprising genes for insecticidal proteins are for example, but not excluding others, MON87701, MON87751 and DAS-81419.

Transgenic cotton events comprising genes for insecticidal proteins are for example, but not excluding others, SGK321, MON531, MON757, MON1076, MON15985, 31707, 31803, 31807, 31808, 42317, BNLA-601, Event1, COT67B, COT102, T303-3, T304-40, GFM Cry1A, GK12, MLS 9124, 281-24-236, 3006-210-23, GHB119 and SGK321.

Increased yield has been created by increasing ear biomass using the transgene athb17, being present in corn event MON87403, or by enhancing photosynthesis using the transgene bbx32, being present in the soybean event MON87712.

Crops comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, in particular to tolerance to drought, has been created by using the transgene cspB, comprised by the corn event MON87460 and by using the transgene Hahb4, comprised by soybean event IND-ØØ41Ø-5.

Traits are frequently combined by combining genes in a transformation event or by combining different events during the breeding process. Preferred combination of traits are herbicide tolerance to different groups of herbicides, insect tolerance to different kind of insects, in particular tolerance to lepidopteran and coleopteran insects, herbicide tolerance with one or several types of insect resistance, herbicide tolerance with increased yield as well as a combination of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-gmc.org/GMCropDatabase), as well as in patent applications, like EP3028573 and WO2017/011288.

The use of the compounds of formula (I) or formulations or combinations comprising them according to the invention on crops may result in effects which are specific to a crop comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

Furthermore, plants are also covered that contain by the use of recombinant DNA techniques a modified amount of ingredients or new ingredients, specifically to improve raw material production, e.g., potatoes that produce increased amounts of amylopectin (e.g. Amflora® potato, BASF SE, Germany).

Furthermore, it has been found that the phenyluracils of formula (I) according to the invention, or the formulations comprising them, are also suitable for the defoliation and/or desiccation of plant parts of crops such as cotton, potato, oilseed rape, sunflower, soybean or field beans, in particular cotton. In this regard, formulations for the desiccation and/or defoliation of crops, processes for preparing these formulations and methods for desiccating and/or defoliating plants using the phenyluracils of formula (I) have been found.

As desiccants, the phenyluracils of formula (I) are particularly suitable for desiccating the aboveground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pernicious fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

### A PREPARATION EXAMPLES

### Example 1.1

### Ethyl 2-[[3-[2-chloro-5-[4-(1,1-difluoroethyl)-3-methyl-2,6-dioxo-pyrimidin-1-yl]-4-fluoro-phenoxy]-2-pyridyl]oxy]acetate

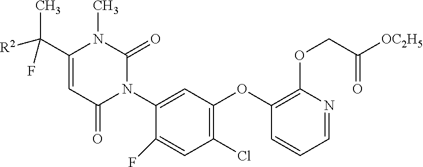

### Example 1.1—Step 1

### Ethyl 4,4-difluoro-3-oxo-pentanoate

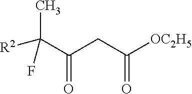

To a solution of commercially available ethyl 2,2-difluoropropanoate (CAS 28781-85-3; 114 g, 826.1 mmol) in tetrahydrofurane (THF) was added ethyl acetate (87.6 g, 991.3 mmol) at 15° C., then NaH (39.7 g, 991.3 mmol) was added at 0° C. The mixture was stirred at 15° C. for 16 h. The mixture was poured into water, extracted with ethyl acetaet, the organic layers were washed with brine, dried over Na₂SO₄, filtered and concentrated to give ethyl 4,4-difluoro-3-oxo-pentanoate (140 g, crude) as brown oil, which was used directly in the next step.

### Example 1.1—Step 2

### Ethyl (Z)-3-amino-4,4-difluoro-pent-2-enoate

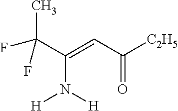

To a solution of ethyl 4,4-difluoro-3-oxo-pentanoate (140 g, 778 mmol) in ethanol and H₂O was added ammonium acetate (180 g, 2333 mmol) at 15° C. and stirred at 90° C. for 16 h. The mixture was poured into water and extracted with ethyl acetate. The organic layers were washed with brine, dried and concentrated to give ethyl (Z)-3-amino-4,4-difluoro-pent-2-enoate (120 g, crude) as brown oil, which was used directly in the next step.

### Example 1.1—Step 3

### 6-(1,1-difluoroethyl)-1H-pyrimidine-2,4-dione

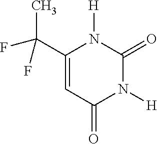

To a solution of ethyl (Z)-3-amino-4,4-difluoro-pent-2-enoate (30 g, 167.6 mmol) in dimethylformamide (DMF), was added KNCO (203.6 g, 2514 mmol) and stirred at 140° C. for 96 h. The mixture was concentrated to remove DMF, the crude was triturated with methyl-tert-butyl-ether (MTBE), filtered, then poured into water, the aqueous phase was adjusted to pH<1, filtered and the aqueous phase extracted with ethyl acetate. The organic layers were washed with brine, dried over Na₂SO₄, filtered and concentrated to give 6-(1,1-difluoroethyl)-1H-pyrimidine-2,4-dione (17 g, 57.6%) as brown solid.

¹H NMR (400 MHZ, DMSO): δ ppm=11.42 (s, 1H), 11.37 (s, 1H), 5.71 (s, 1H), 1.94 (t, 3H)

### Example 1.1—Step 4

### 6-(1,1-difluoroethyl)-1-methyl-pyrimidine-2,4-dione

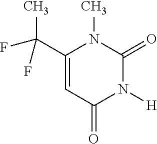

To a solution of 6-(1,1-difluoroethyl)-1H-pyrimidine-2,4-dione (17 g, 96.6 mmol) in THF was added potassium tert-butanolate (t-BuOK) (13 g, 115.9 mmol) and CF₃SO₃CH₃ (19.3 g, 115.9 mmol) slowly at 0° C. under N₂. The mixture was subsequently stirred at 15° C. for 4 h. The mixture was concentrated to remove THF, the crude was poured into water (500 ml), the aqueous phase adjusted to pH=7, extracted with ethyl acetate, the organic layers were washed with brine, dried over Na₂SO₄, filtered and concentrated to give 6-(1,1-difluoroethyl)-1-methyl-pyrimidine-2,4-dione (13 g, crude) as yellow solid, which was used directly in the next step.

### Example 1.1—Step 5

### 6-(1,1-difluoroethyl)-3-(2,5-difluoro-4-nitro-phenyl)-1-methyl-pyrimidine-2,4-dione

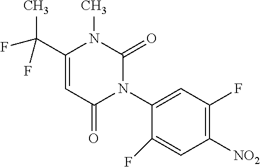 [SMILES: Cn1c(C(C)(F)F)cc(=O)n(-c2cc(F)c([N+](=O)[O-])cc2F)c1=O]

To a mixture of 6-(1,1-difluoroethyl)-1-methyl-pyrimidine-2,4-dione (3.5 g, 18.4 mmol) in DMF (40 mL) was added 1,2,4-trifluoro-5-nitro-benzene (3.9 g, 22 mmol) and Cs₂CO₃ (7.7 g, 22 mmol) at 20° C. The mixture was stirred at 80° C. for 16 h under N₂. The mixture was poured into water and extracted with ethyl acetate. The organic layer was washed with brine, dried over Na₂SO₄, concentrated and purified by column chromatography (ethyl acetate in petrol ether; increase of ethyl acetate part from 0% to 100%) to give 6-(1,1-difluoroethyl)-3-(2,5-difluoro-4-nitro-phenyl)-1-methyl-pyrimidine-2,4-dione (4.7 g, 73.6%).

¹H NMR (CDCl₃ 400 MHz): δ ppm=7.99 (dd, 1H), 7.32 (dd, 1H), 6.18 (s, 1H), 3.60 (s, 3H), 2.08 (t, 3H)

### Example 1.1—Step 6

### Ethyl 2-[[3-[5-[4-(1,1-difluoroethyl)-3-methyl-2,6-dioxo-pyrimidin-1-yl]-4-fluoro-2-nitro-phenoxy]-2-pyridyl]oxy]acetate

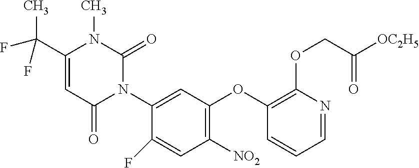 [SMILES: CCOC(=O)COc1ncccc1Oc1cc(-n2c(=O)cc(C(C)(F)F)n(C)c2=O)c(F)cc1[N+](=O)[O-]]

To a solution of 6-(1,1-difluoroethyl)-3-(2,5-difluoro-4-nitro-phenyl)-1-methyl-pyrimidine-2,4-dione (3.47 g, 10 mol) in DMF, was added ethyl 2-[(3-hydroxy-2-pyridyl)oxy]acetate (3 g, 15 mol) and K₂CO₃ (2.76 g, 20 mol). The resulting mixture was stirred at 15° C. for 12 h. The mixture was poured into water and extracted with ethyl acetate. The organic layers were washed with brine, dried over Na₂SO₄, filtered and concentrated to give crude ethyl 2-[[3-[5-[4-(1,1-difluoroethyl)-3-methyl-2,6-dioxo-pyrimidin-1-yl]-4-fluoro-2-nitro-phenoxy]-2-pyridyl]oxy]acetate (5.2 g, crude), which was used directly in the next step.

### Example 1.1—Step 7

### Ethyl 2-[[3-[2-amino-5-[4-(1,1-difluoroethyl)-3-methyl-2,6-dioxo-pyrimidin-1-yl]-4-fluoro-phenoxy]-2-pyridyl]oxy]acetate

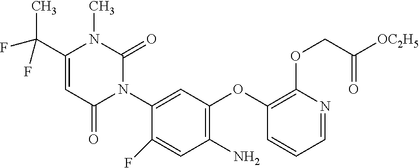 [SMILES: CCOC(=O)COc1ncccc1Oc1cc(-n2c(=O)cc(C(C)(F)F)n(C)c2=O)c(F)cc1N]

To a solution of ethyl 2-[[3-[5-[4-(1,1-difluoroethyl)-3-methyl-2,6-dioxo-pyrimidin-1-yl]-4-fluoro-2-nitro-phenoxy]-2-pyridyl]oxy]acetate (5.2 g, 10 mmol) in 40 ml ethanol was added H₂O, Fe (2.8 g, 50 mmol) and NH₄Cl (2.7 g, 50 mmol) at 15° C. The mixture was stirred at 80° C. for 1 h. The mixture was then filtered, concentrated and purified by column chromatography to give ethyl 2-[[3-[2-amino-5-[4-(1,1-difluoroethyl)-3-methyl-2,6-dioxo-pyrimidin-1-yl]-4-fluoro-phenoxy]-2-pyridyl]oxy]acetate (4 g, 80%) as a yellow oil, which was used directly in the next step.

### Example 1.1—Step 8

### Ethyl 2-[[3-[2-chloro-5-[4-(1,1-difluoroethyl)-3-methyl-2,6-dioxo-pyrimidin-1-yl]-4-fluoro-phenoxy]-2-pyridyl]oxy]acetate

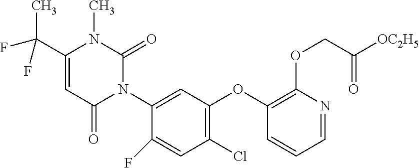 [SMILES: CCOC(=O)COc1ncccc1Oc1cc(-n2c(=O)cc(C(C)(F)F)n(C)c2=O)c(F)cc1Cl]

To a mixture of ethyl 2-[[3-[2-amino-5-[4-(1,1-difluoroethyl)-3-methyl-2,6-dioxo-pyrimidin-1-yl]-4-fluoro-phenoxy]-2-pyridyl]oxy]acetate (4 g, 8 mmol) in acetonitrile was added CuCl (4 g, 40 mmol), CuCl₂ (5.4 g, 40 mmol) and tert-butyl nitrite (4 g, 40 mmol) at 15° C. The mixture was stirred at 15° C. for 30 min. The mixture was poured into water and extracted with ethyl acetate. The organic layer was washed with brine, dried over anhydrous Na₂SO₄, concentrated and purified by preparative HPLC (acetonitrile-H₂O with trifluoroacetic acid) to give ethyl 2-[[3-[2-chloro-5-[4-(1,1-difluoroethyl)-3-methyl-2,6-dioxo-pyrimidin-1-yl]-4-fluoro-phenoxy]-2-pyridyl]oxy]acetate (2.2 g, 53.6%) as a yellow solid.

¹H NMR (CDCl₃ 400 MHz): δ ppm=7.91 (dd, 1H), 7.37 (d, 1H), 7.29 (dd, 1H), 6.98-6.85 (m, 2H), 6.09 (s, 1H), 4.93 (s, 2H), 4.18 (q, 2H), 3.54 (s, 3H), 2.03 (t, 3H), 1.25 (t, 3H).

### Example 1.2

### Ethyl 2-[[3-[2-chloro-5-[4-(1,1-difluoroethyl)-3-methyl-2,6-dioxo-pyrimidin-1-yl]-4-fluoro-phenoxy]-2-phenoxy]acetate

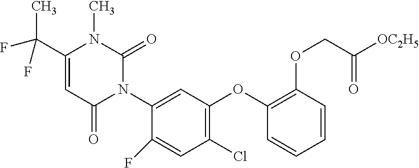 [SMILES: CCOC(=O)COc1ccccc1Oc1cc(-n2c(=O)cc(C(C)(F)F)n(C)c2=O)c(F)cc1Cl]

### Example 1.2—Step 1

### 6-(1,1-difluoroethyl)-3-[2-fluoro-5-(2-methoxyphenoxy)-4-nitro-phenyl]-1-methyl-pyrimidine-2,4-dione

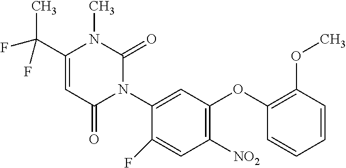 [SMILES: COc1ccccc1Oc1cc(-n2c(=O)cc(C(C)(F)F)n(C)c2=O)c(F)cc1[N+](=O)[O-]]

To a solution of 6-(1,1-difluoroethyl)-3-(2,5-difluoro-4-nitro-phenyl)-1-methyl-pyrimidine-2,4-dione (3.47 g, 10 mmol, s. Example 1—Step 5) in 50 ml acetonitrile was added 2-methoxyphenol (1.4 g, 11 mmol) and K₂CO₃ (6.5 g, 20 mmol). The resulting mixture was stirred at 20° C. for 16 hours. The mixture was poured into water, extracted with ethylacetate, the organic layers were washed with brine, dried over Na₂SO₄, filtered and concentrated to give 6-(1,1-difluoroethyl)-3-[2-fluoro-5-(2-methoxyphenoxy)-4-nitro-phenyl]-1-methyl-pyrimidine-2,4-dione (10 g, crude), which was used directly in the next step.

### Example 1.2—Step 2

### 3-[4-amino-2-fluoro-5-(2-methoxyphenoxy)phenyl]-6-(1,1-difluoroethyl)-1-methyl-pyrimidine-2,4-dione

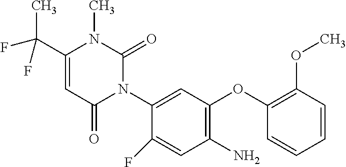 [SMILES: COc1ccccc1Oc1cc(-n2c(=O)cc(C(C)(F)F)n(C)c2=O)c(F)cc1N]

To a solution of 6-(1,1-difluoroethyl)-3-[2-fluoro-5-(2-methoxyphenoxy)-4-nitro-phenyl]-1-methylpyrimidine-2,4-dione (10 g, 22 mmol) in 80 ml ethanol was added water, Fe (6.21 g, 111 mmol) and NH₄Cl (5.9 g, 111 mmol) at 15° C. The mixture was stirred at 80° C. for 1 hour, filtered, concentrated and purified by column chromatography (ethyl acetate in petrol ether; increase of ethyl acetate part from 0% to 100%) to give 3-[4-amino-2-fluoro-5-(2-methoxyphenoxy)phenyl]-6-(1,1-difluoroethyl)-1-methyl-pyrimidine-2,4-dione (7 g, 75%) as a yellow solid.

¹H NMR (CDCl₃ 400 MHz): δ ppm=7.16-6.85 (m, 4H), 6.69-6.51 (m, 2H), 6.08 (s, 1H), 4.20 (br s, 2H), 3.85 (s, 3H), 3.54 (s, 3H), 2.01 (t, 3H)

### Example 1.2—Step 3

### 3-[4-chloro-2-fluoro-5-(2-methoxyphenoxy)phenyl]-6-(1,1-difluoroethyl)-1-methyl-pyrimidine-2,4-dione

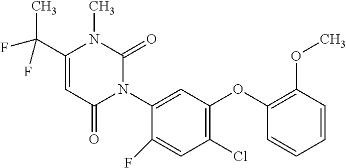 [SMILES: COc1ccccc1Oc1cc(-n2c(=O)cc(C(C)(F)F)n(C)c2=O)c(F)cc1Cl]

To a mixture of 3-[4-amino-2-fluoro-5-(2-methoxyphenoxy)phenyl]-6-(1,1-difluoroethyl)-1-methylpyrimidine-2,4-dione (7 g, 16.6 mmol) in acetonitrile was added CuCl (8.3 g, 83 mmol), CuCl2 (11.2 g, 83 mmol) and tert-butyl nitrite (8.3 g, 83 mmol) at 15° C. The mixture was stirred at 15° C. for 30 min. and then poured into water and extracted with ethyl acetate. The organic layer was washed with brine, dried over anhydrous Na₂SO₄, concentrated and purified by column chromatography (ethyl acetate in petrol ether; increase of ethyl acetate part from 0% to 15%) to give 3-[4-chloro-2-fluoro-5-(2-methoxyphenoxy)phenyl]-6-(1,1-difluoroethyl)-1-methylpyrimidine-2,4-dione (7.2 g, crude).

¹H NMR (CDCl₃ 400 MHz): δ ppm=7.36 (d, J=8.9 Hz, 1H), 7.21-7.12 (m, 1H), 7.07-6.89 (m, 3H), 6.63 (d, J=6.6 Hz, 1H), 6.09 (s, 1H), 3.82 (s, 3H), 3.54 (s, 3H), 2.01 (t, 3H)

### Example 1.2—Step 4

### 3-[4-chloro-2-fluoro-5-(2-hydroxyphenoxy)phenyl]-6-(1,1-difluoroethyl)-1-methyl-pyrimidine-2,4-dione

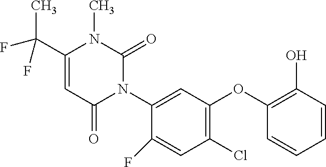 [SMILES: Cn1c(C(C)(F)F)cc(=O)n(-c2cc(Oc3ccccc3O)c(Cl)cc2F)c1=O]

To a mixture of 3-[4-chloro-2-fluoro-5-(2-methoxyphenoxy)phenyl]-6-(1,1-difluoroethyl)-1-methylpyrimidine-2,4-dione (7.2 g, 16.4 mmol) in dichloromethane was added BBr₃ (8.2 g, 32.7 mmol) at 0° C. The mixture was stirred at 20° C. for 1 hour and then poured into water and extracted with dichloromethane. The organic layer was washed with brine, dried over anhydrous Na₂SO₄ and concentrated to give 3-[4-chloro-2-fluoro-5-(2-hydroxyphenoxy)phenyl]-6-(1,1-difluoroethyl)-1-methyl-pyrimidine-2,4-dione (6 g, 86%), which was used directly in the next step.

### Example 1.2—Step 5

### Ethyl 2-[[3-[2-chloro-5-[4-(1,1-difluoroethyl)-3-methyl-2,6-dioxo-pyrimidin-1-yl]-4-fluoro-phenoxy]-2-phenoxy]acetate

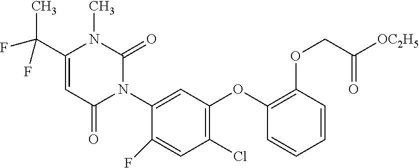 [SMILES: CCOC(=O)COc1ccccc1Oc1cc(-n2c(=O)cc(C(C)(F)F)n(C)c2=O)c(F)cc1Cl]

To a mixture of 3-[4-chloro-2-fluoro-5-(2-hydroxyphenoxy)phenyl]-6-(1,1-difluoroethyl)-1-methylpyrimidine-2,4-dione (6 g, 14.08 mmol) in acetonitrile was added K₂CO₃ (3.9 g, 28.2 mmol) and ethyl 2-bromoacetate (3.53 g, 21.1 mmol) at 20° C. The mixture was stirred at 20° C. for 16 hours, then poured into water and extracted with ethyl acetate. The organic layer was washed with brine, dried over anhydrous Na₂SO₄, concentrated and purified by column chromatography (ethyl acetate in petrol ether; increase of ethyl acetate part from 0% to 15%) to give ehyl 2-[[3-[2-chloro-5-[4-(1,1-difluoroethyl)-3-methyl-2,6-dioxo-pyrimidin-1-yl]-4-fluoro-phenoxy]-2-phenoxy]acetate (4.09 g, 56.7%).

¹H NMR (CDCl₃ 400 MHz): 0 ppm=7.36 (d, J=8.9 Hz, 1H), 7.15-7.09 (m, 1H), 7.08-7.03 (m, 1H), 7.02-6.96 (m, 1H), 6.93 (d, J=8.1 Hz, 1H), 6.77 (d, J=6.6 Hz, 1H), 6.08 (s, 1H), 4.65 (s, 2H), 4.21 (q, J=7.1 Hz, 2H), 3.53 (s, 3H), 2.01 (t, J=18.8 Hz, 3H), 1.26 (t, J=7.1 Hz, 3H)

The compounds listed below in tables 1 and 2 can be prepared similarly to the example mentioned above.

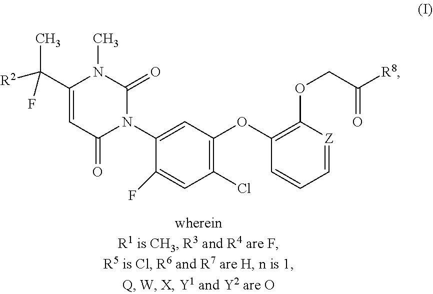

TABLE 1

| no | R² | R⁸ | Z | m/z [M + H] | Rt [min] |
|---|---|---|---|---|---|
| 1.1 | F | OC₂H₅ | N | 514 | 1.238 |
| 1.2 | F | OC₂H₅ | CH | 513 | 1.257 |
| 1.3 | F | OH | CH | 485 | 1.095 |
| 1.4 | F | OH | N | 486 | 1.063 |
| 1.5 | H | OC₂H₅ | CH | 517* | 1.196 |
| 1.6 | F | OC₂H₄OCH₃ | N | 544 | 1.179 |
| 1.7 | F | OCH₂-2-tetrahydrofuranyl | N | 570 | 1.214 |
| 1.8 | F | OCH₂CN | N | 525 | 1.162 |

*m/z [M + Na]

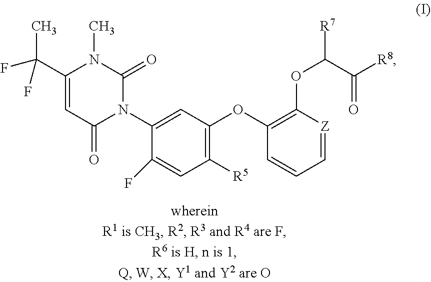

TABLE 2

| no | R⁵ | R⁷ | R⁸ | Z | m/z [M + H] | Rt [min] |
|---|---|---|---|---|---|---|
| 2.1 | Cl | OCH₃ | OCH₃ | CH | 529 | 1.221 |
| 2.2 | Br | OCH₃ | OCH₃ | CH | 573 | 1.229 |
| 2.3 | Br | H | OH | N | 530 | 1.071 |
| 2.4 | Br | H | OC₂H₅ | N | 558 | 1.236 |
| 2.5 | Br | H | OC₂H₄OCH₃ | N | 588 | 1.190 |
| 2.6 | Br | H | OCH₂-2- | N | 614 | 1.225 |
| | | | tetrahydrofuranyl | | | |

### B USE EXAMPLES

The herbicidal activity of the phenyluracils of formula (I) was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the test plants had rooted. This cover caused uniform germination of the test plants, unless this had been impaired by the active ingredients. For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the test plants were kept at 10-25° C. or 20-35° C., respectively. The test period extended over 2 to 3 weeks. During this time, the test plants were tended, and their response to the individual treatments was evaluated.

Evaluation was carried out using a scale from 0 to 100. 100 means no emergence of the test plants, or complete destruction of at least the aerial moieties, and 0 means no damage, or normal course of growth. A good herbicidal activity is given at values of at least 70 and a very good herbicidal activity is given at values of at least 85.

The test plants used in the greenhouse experiments were of the following species:

| Bayer code | Scientific name |
|---|---|
| AMARE | Amaranthus retroflexus |
| CHEAL | Chenopodium album |
| ERICA | Erigeron canadensis |
| POLCO | Polygonum convolvulus |
| SETFA | Setaria faberi |
| SETVI | Setaria viridis |
| TRCIN | Digitaria insularis |

At an application rate of 16 g/ha, the compounds (examples) 1.1, 1.2 and 1.3 applied post-emergent, showed very good herbicidal activity against AMARE, CHEAL, POLCO and SETVI.

At an application rate of 16 g/ha, the compounds (examples) 1.6, 1.7, 2.5 and 2.6 applied pre-emergent, showed very good herbicidal activity against AMARE, POLCO and SETVI.

At an application rate of 16 g/ha, the compounds (examples) 1.8, 2.3 and 2.4 applied pre-emergent, showed very good herbicidal activity against AMARE and SETVI.

At an application rate of 16 g/ha, the compounds (examples) 2.1 and 2.2 applied pre-emergent, showed very good herbicidal activity against AMARE, CHEAL, POLCO and SETVI.

Tables 3, 4 and 5: Comparison of the herbicidal activity of compound I-2-8 known from EP 1 122 244 (8th compound Table 2 page 75) and example 1.1 of the present invention:

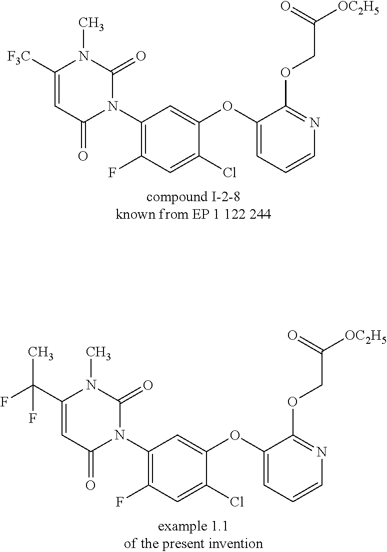 [SMILES: CCOC(=O)COc1ncccc1Oc1cc(-n2c(=O)cc(C(F)(F)F)n(C)c2=O)c(F)cc1Cl ; CCOC(=O)COc1ncccc1Oc1cc(-n2c(=O)cc(C(C)(F)F)n(C)c2=O)c(F)cc1Cl]

TABLE 3
(post-emergence; greenhouse)

| compound | cmpd I-2-8 (EP 1 122 244) | example 1.1 |
|---|---|---|
| application rate [g/ha] | 4 | 4 |
| | damages | |
| unwanted plants | | |
| ERICA | 65 | 100 |
| SETFA | 90 | 95 |
| TRCIN | 90 | 100 |

TABLE 4
(post-emergence; greenhouse)

| compound | cmpd I-2-8 (EP 1 122 244) | example 1.1 |
|---|---|---|
| application rate [g/ha] | 2 | 2 |
| | damages | |
| unwanted plants | | |
| SETFA | 85 | 90 |
| TRCIN | 90 | 95 |

TABLE 5
(post-emergence; greenhouse)

| compound | cmpd I-2-8 (EP 1 122 244) | example 1.1 |
|---|---|---|
| application rate [g/ha] | 1 | 1 |
| | damages | |
| unwanted plants | | |
| SETFA | 70 | 80 |

The replacement of one fluorine atom at the substituent attached in 4-position of the uracil ring (known from literature) by a methyl group according to the invention leads to a better herbicidal activity, not only at higher, but also at lower application rates compared to the results achieved by the compound I-2-8 known from EP 1 122 244.

1. A phenyluracil of formula (I) 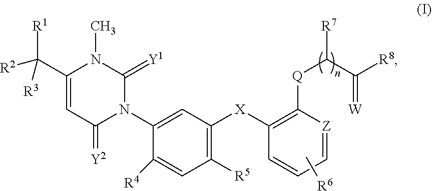
wherein the substituents have the following meanings:
R¹ CH₃;
R² halogen;
R³ halogen;
R⁴ halogen;
R⁵ halogen;
R⁶ H;
R⁷ H or C₁-C₃-alkoxy;
R⁸ OR⁹, wherein
R⁹ is hydrogen or C₁-C₆-alkyl,
n 1;
Q, W, X, Y¹, Y² O;
Z CH or N;
including agriculturally acceptable salts, esters selected from the group consisting of 1-methylhexyl, heptyl, octyl, 2-ethylhexyl, 2-methoxyethyl, 2-ethoxyethyl, 2-butoxyethyl, 2-butoxypropyl and 3-butoxypropyl esters, and C₁-C₁₀-alkylthio esters,
provided the compound of formula (I) has a carboxyl group.

2. The phenyluracil of formula (I) according to claim 1 wherein R² and R³ are F.

3. The phenyluracil of formula (I) according to claim 1, wherein R⁴ is F.

4. The phenyluracil of formula (I) according to claim 1, wherein R⁵ is Cl or Br.

5. The phenyluracil of formula (I) according to claim 1, wherein R⁷ is H or OCH₃.

6. The phenyluracil of formula (I) according to claim 1, wherein R⁷ is C₁-C₃-alkoxy.

7. The phenyluracil of formula (I) according to claim 1 wherein R⁹ is hydrogen, CH₃, or C₂H₅.

8. The phenyluracil of formula (I) according to claim 1, wherein Z is CH.

9. The phenyluracil of formula (I) according to claim 1, wherein Z is N.

10. An herbicidal composition comprising an herbicidally active amount of at least one phenyluracil of formula (I) as claimed in claim 1 and at least one inert liquid and/or solid carrier and, optionally, at least one surface-active substance.

11. A process for a preparation of an herbicidal active composition, comprising mixing an herbicidally active amount of at least one phenyluracil of formula (I) as claimed in claim 1 and at least one inert liquid and/or solid carrier and, optionally, at least one surface-active substance.

12. A method of controlling undesired vegetation, comprising contacting an herbicidally active amount of at least one phenyluracil of formula (I) as claimed in claim 1 with plants, their environment, or on seed.

13. (canceled)
